(19) United States
(12) Patent Application Publication
(10) Pub. No.: US 2014/0155362 A1
(43) Pub. Date: Jun. 5, 2014

(54) BHQ-CONJUGATES, AND RELATED COMPOUNDS, METHODS OF MAKING THE SAME, AND METHODS OF USE THEREOF

(71) Applicants: Timothy M. Dore, Athens, GA (US); James D. Lauderdale, Statham, GA (US); Adam C. Rea, Athens, GA (US); Adna Muliawan, Alpharetta, GA (US); Duncan McLain, Atens, GA (US)

(72) Inventors: Timothy M. Dore, Athens, GA (US); James D. Lauderdale, Statham, GA (US); Adam C. Rea, Athens, GA (US); Adna Muliawan, Alpharetta, GA (US); Duncan McLain, Atens, GA (US)

(21) Appl. No.: 14/117,058

(22) PCT Filed: Jun. 28, 2012
(86) PCT No.: PCT/US12/44567

§ 371 Date: Nov. 12, 2013

Related U.S. Application Data
(60) Provisional application No. 61/501,967, filed on Jun. 28, 2011.
(60) Provisional application No. 61/511,586, filed on Jul. 26, 2011.

(51) Int. Cl. A61K 47/48; A61N 5/06; A61K 31/4045; A61K 31/138; A61K 31/58; A61K 31/165

(52) U.S. Cl. CPC A61K 47/48061; A61K 31/58; A61K 31/165; A61K 31/4045; A61K 31/138; A61N 5/062
USPC 514/176; 514/282; 514/311; 514/314; 540/113; 546/46; 546/167; 546/174; 546/177; 604/20

(57) ABSTRACT

Embodiments of the present disclosure provide for BHQ-conjugates and protected BHQ-conjugate precursor compounds, methods of making BHQ-conjugates and protected BHQ-conjugate precursor compounds, methods of using BHQ-conjugates and protected BHQ-conjugate precursor compounds, and the like.

### CROSS-REFERENCE TO RELATED APPLICATION

This application claims priority to U.S. provisional application entitled “BHQ-O-5HT, BHQ-N-5HT, AND RELATED COMPOUNDS, METHODS OF MAKING THE SAME, AND METHODS OF USE THEREOF,” having Ser. No. 61/501,967, filed on Jun. 28, 2011, which is entirely incorporated herein by reference. In addition, this application claims priority to U.S. provisional application entitled “BHQ-VNA, BHQ-VAA, AND RELATED COMPOUNDS, METHODS OF MAKING THE SAME, AND METHODS OF USE THEREOF,” having Ser. No. 61/511,586, filed on Jul. 26, 2011, which is entirely incorporated herein by reference.

### STATEMENT REGARDING FEDERALLY SPONSORED RESEARCH OR DEVELOPMENT

This invention(s) was made with government support under Grant Nos.: R01 NS070159 and CHE-1012412, awarded by the National Institutes of Health and the National Science Foundation, respectively. The government has certain rights in the invention(s).

### BACKGROUND

Biologically active compounds with a phenol functional group often play a role in mediating important physiological processes, including neurotransmission and embryonic development. Controlling the release of these compounds is critical for understanding their physiological function. Thus, there is a need to find compounds that can controllably release biologically active compounds

### SUMMARY

Embodiments of the present disclosure provide for BHQ-conjugates and protected BHQ-conjugate precursor compounds, methods of making BHQ-conjugates and protected BHQ-conjugate precursor compounds, methods of using BHQ-conjugates and protected BHQ-conjugate precursor compounds, and the like.

An exemplary embodiment of the composition, among others, includes: a BHQ-conjugate or a protected BHQ-conjugate precursor compound. In an embodiment, the conjugate can include a biologically active compound including a phenol group.

In an embodiment, the BHQ-conjugate has the following structure:

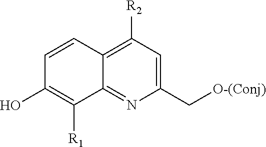

wherein R₁ is selected from the group consisting of: H, Br, F, Cl, I, and CN; and wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl.

In an embodiment, the protected BHQ-conjugate precursor compound has the following structure:

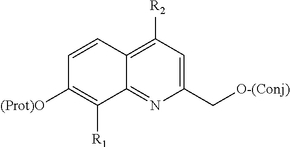

wherein R₁ is selected from the group consisting of: H, Br, F, Cl, I, and CN; wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl; wherein the Prot group is selected from the group consisting of: a methoxymethyl ether (MOM) group, a β-methoxyethoxymethyl ether (MEM) group, a methyl group (Me), a methyl thiomethyl (MTM) group, a benzyloxymethyl (BOM) group, a tetrahydropyranyl (THP) group, an ethoxyethyl (EE) group, a trityl (Tr) group, a methoxytrityl group, a benzene sulfonyl (Bs) group, a toluenesulfonyl (Ts) group, and a silicon-based protecting group.

An exemplary embodiment of a method of treating a condition, among others, includes: administering a pharmaceutically effective amount of BHQ-conjugate to a subject in need of treatment.

An exemplary embodiment of a method of releasing a conjugate, among others, includes: exposing a BHQ-conjugate to a light energy, wherein the light energy interacts with the BHQ-conjugate and causes the conjugate to be released from the BHQ-conjugate.

An exemplary embodiment of a pharmaceutical composition, among others, includes: a pharmaceutically effective amount of a BHQ-conjugate.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1.1 illustrates an electrophysiological response to photochemical release of 5HT from BHQ-O-5HT in ex vivo zebrafish brain.

FIG. 2.1 illustrates an electrophysiological response to photochemical release of VNA, a capsaicin analog, from BHQ-VNA on cultured dorsal root ganglia cells prepared from an adult mouse.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. Terms defined in references that are incorporated by reference do not alter definitions of terms defined in the present disclosure or should such terms be used to define terms in the present disclosure they should only be used in a manner that is inconsistent with the present disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, inorganic chemistry, material science, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

By “administration” is meant introducing a composition of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “an effective amount” of the composition of the present disclosure is defined as an amount sufficient to yield an acceptable outcome (treatment of the condition or disease). In an embodiment, an effective amount of the composition of the present disclosure may be administered in more than one injection or stimulation. The effective amount of the compositions of the present disclosure can vary according to factors such as the degree of susceptibility of the individual, the age, sex, and weight of the individual, idiosyncratic responses of the individual, the dosimetry, and the like.

As used herein, “treat”, “treatment”, “treating”, and the like refer to acting upon a disease, condition, or disorder with a composition to affect the disease, condition, or disorder by improving or altering it. The improvement or alteration may include an improvement in symptoms or an alteration in the physiologic pathways associated with the disease, condition, or disorder. “Treatment,” as used herein, covers one or more treatments of a disease in a host (e.g., a mammal, typically a human or non-human animal of veterinary interest), and includes: (a) reducing the risk of occurrence of the disease, condition, or disorder in a subject determined to be predisposed to the disease but not yet diagnosed as infected with the disease, condition, or disorder, (b) impeding the development of the disease, condition, or disorder, and/or (c) relieving the disease, condition, or disorder, e.g., causing regression of the disease, condition, or disorder and/or relieving one or more disease, condition, or disorder symptoms.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a disease, condition, or disorder or symptom thereof and/or may be therapeutic in terms of a partial or complete cure for a disease, condition, or disorder and/or adverse effect attributable to the disease, condition, or disorder.

The term “unit dosage form,” as used herein, refers to physically discrete units suitable as unitary dosages for human and/or animal subjects, each unit containing a predetermined quantity of a composition calculated in an amount sufficient (e.g., weight of host, disease, severity of the disease, etc) to produce the desired effect. The specifications for unit dosage forms depend on the particular composition employed, the route and frequency of administration, and the effect to be achieved, and the pharmacodynamics associated with the composition in the host.

The term “therapeutically effective amount” as used herein refers to that amount of an embodiment of the composition being administered that will relieve to some extent one or more of the symptoms of the disease, condition, or disorder being treated, and/or that amount that will prevent, to some extent, one or more of the symptoms of the disease, condition, or disorder that the host being treated has or is at risk of developing.

As used herein, the term “subject” or “host” includes humans and mammals (e.g., mice, rats, pigs, cats, dogs, and horses,). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to host or organisms noted above that are alive. The term “living subject” refers to the entire host or organism and not just a part excised (e.g., a liver or other organ) from the living subject.

The term “substituted” refers to any one or more hydrogens on the designated atom that can be replaced with a selection from the indicated group, provided that the designated atom's normal valence is not exceeded, and that the substitution results in a stable compound.

The terms “alk” or “alkyl” refer to straight or branched chain hydrocarbon groups having 1 to 24 carbon atoms, preferably 6 to 18 carbon atoms, such as methyl, ethyl, n-propyl, i-propyl, n-butyl, i-butyl, t-butyl, pentyl, hexyl, heptyl, n-octyl, dodecyl, octadecyl, amyl, 2-ethylhexyl, and the like. An alkyl group is optionally substituted, unless stated otherwise, with one or more groups, selected from aryl (optionally substituted), heterocyclo (optionally substituted), carbocyclo (optionally substituted), halo, hydroxy, protected hydroxy, alkoxy (e.g., C₁ to C₇) (optionally substituted), acyl (e.g., C₁ to C₇), aryloxy (e.g., C₁ to C₇) (optionally substituted), alkylester (optionally substituted), arylester (optionally substituted), alkanoyl (optionally substituted), aroyl (optionally substituted), carboxy, protected carboxy, cyano, nitro, amino, substituted amino, (monosubstituted)amino, (disubstituted)amino, protected amino, amido, carbamate, lactam, urea, urethane, sulfonyl, and the like. In an embodiment, the amino group is a protected amino group.

The terms “ar” or “aryl” refer to aromatic homocyclic (i.e., hydrocarbon) mono-, bi- or tricyclic ring-containing groups preferably having 6 to 12 members such as phenyl, naphthyl and biphenyl. An aryl group is optionally substituted, unless stated otherwise, with one or more groups, selected from alkyl (optionally substituted alkyl), alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, carbamate, lactam, urea, urethane, sulfonyl, and the like. Optionally, adjacent substituents, together with the atoms to which they are bonded, form a 3- to 7-member ring. In an embodiment, the amino group is a protected amino group.

The term “heteroaryl” refers to optionally substituted five-membered or six-membered rings that have 1 to 4 heteroatoms, such as oxygen, sulfur and/or nitrogen atoms, either alone or in conjunction with, additional nitrogen, sulfur or oxygen ring atoms. Furthermore, the above optionally substituted five-membered or six-membered rings can optionally be fused to an aromatic 5-membered or 6-membered ring system. For example, the rings can be optionally fused to an aromatic 5-membered or 6-membered ring system such as a benzene, pyridine or a triazole system.

### Discussion

Embodiments of the present disclosure provide for BHQ-conjugates and protected BHQ-conjugate precursor compounds, methods of making BHQ-conjugates and protected BHQ-conjugate precursor compounds, methods of using BHQ-conjugates and protected BHQ-conjugate precursor compounds, and the like. In an embodiment, the conjugate can include biologically active compounds including a phenol group. Additional details regarding the compounds and methods of making are described in the Example.

An embodiment of the present disclosure includes a BHQ-conjugate or a protected BHQ-conjugate precursor compound, where each can include multiple isomers. In addition, the conjugate can be attached to the BHQ at different points of the conjugate (See BHQ-O-serotonin and BHQ-N-serotonin). In an embodiment, the BHQ-conjugate can have the following structure:

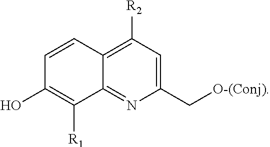

In an embodiment, R₁ can include: H, Br, F, Cl, I, or CN. In an embodiment, R₂ can include: H, F, Cl, Br, I, OH, OR, NRR′, CH₃, CN, an unsubstituted or substituted alkyl, or unsubstituted or substituted aryl. In an embodiment, R and R′ can each be independently selected from: H, an unsubstituted or substituted alkyl, or an unsubstituted or substituted aryl.

In an embodiment, the conjugate can include biologically active compounds including any phenol group such as serotonin (5HT), a capsaicinoid, a catechol, and other biologically active compounds including a phenol group. In a specific embodiment, the BHQ-conjugate can be: BHQ-O-5HT, BHQ-N-5HT, BHQ-capsaicin, BHQ-VNA (vanillylamide of n-nonanoic acid), BHQ-VAA (vanillylamide of acetic acid), BHQ-dopamine, BHQ-epinephrine, BHQ-noreepinephrine, BHQ-tyrosine, BHQ-tyrosine(N-Fmoc), BHQ-hydroxytamoxifen, BHQ-morphine, BHQ-oripavine, BHQ-estriol, BHQ-estrone, and BHQ-estradiol, where each could include multiple isomers.

An embodiment of the present disclosure includes a protected BHQ-conjugate, where each can include multiple isomers. In addition, the conjugate can be attached to the BHQ at different points of the conjugate (See BHQ-O-serotonin and BHQ-N-serotonin). In an embodiment, the BHQ-conjugate can have the following structure:

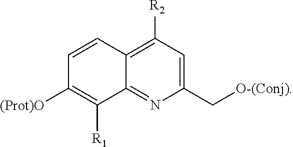

In an embodiment, R₁ can include: H, Br, F, Cl, I, or CN. In an embodiment, R₂ can include: H, F, Cl, Br, I, OH, OR, NRR′, CH₃, CN, an unsubstituted or substituted alkyl, or unsubstituted or substituted aryl. In an embodiment, R and R′ can each be independently selected from: H, an unsubstituted or substituted alkyl, and unsubstituted or substituted aryl.

In an embodiment, the “Prot group” is a protection group for the hydroxyl group on the BHQ compound. In an embodiment, the Prot group can include: a methoxymethyl ether (MOM) group, a β-methoxyethoxymethyl ether (MEM) group, a methyl group (Me), a methyl thiomethyl (MTM) group, a benzyloxymethyl (BOM) group, a tetrahydropyranyl (THP) group, an ethoxyethyl (EE) group, a trityl (Tr) group, a methoxytrityl group, a benzene sulfonyl (Bs) group, a toluenesulfonyl (Ts) group, and a silicon-based protecting group (e.g., t-butyldimethylsilyl(TBS), t-butyldiphenylsilyl (TBDPS), triisopropylsilyl (TIPS), trimethylsilyl (TMS), triethylilyl (TES), dimethylisopropylsilyl (IPDMS), diethylisopropylsilyl (DEIPS), tetraisopropyldisiylene (TIPDS), or di-t-butyldimethylsilylene (DTBS)). Each of the specific BHQ-conjugates mentioned above can include a protecting group (e.g., MOM-BHQ-O-5HT, MOM-BHQ-N-5HT, and the like).

In general, the BHQ-conjugates can be prepared from an appropriate BHQ derivative and an appropriately protected conjugate. A protected BHQ compound such as MOM-BHQ-OH can be used to start the process for forming the BHQ-conjugate. The protected form of the BHQ-conjugate is generally an intermediate, which is deprotected to reveal the BHA-conjugate. In an embodiment, a mesylate can be formed, which is subsequently displaced by a conjugate group (e.g., the conjugate group may include one or more protecting groups such as those described herein) such as Boc-protected 5HT. In one or more steps the compound can be deprotected using techniques known in the art. One or more strategies can be used depending upon the conjugate, if the conjugate includes two or more points to bond with the BHQ compound, the type of protecting group(s), and the like. Details regarding methods of making various BHQ-conjugates are provided in the Examples.

In an embodiment, the conjugate can be released from the BHQ-conjugate by exposing the BHQ-conjugate to a light energy. In an embodiment, the light energy (a photon) can have a wavelength of about 300-425 nm and/or 690-850 nm. In an embodiment, the conjugate can be released from the BHQ-conjugate using a single photon or two photons. In this regard, the BHQ-conjugate can be selectively released at a specific location and/or at a specific time to accomplish a goal (e.g., study of the conjugates interaction, treatment, and the like).

Embodiments of the present disclosure can be used to treat a condition (e.g., state, disease, and the like) in a patient in need treatment by administration of one or more compounds of the present disclosure. As described above, the conjugate can be released from the BHQ-conjugate using light energy.

In an embodiment where the conjugate is serotonin, the condition can include: seizure disorders, improved memory, mood: facilitate feeling of well-being, appetite control, sleep, muscle contractions, wound healing, mediate valve development in growth of heart (for transplantation), pain (serotonin can induce pain), diabetes, and a combination thereof. In addition, BHQ-serotonin can be used to study growth factors, in stem cell research, its effect as a laxative, its role in left-right patterning in embryonic development, or a combination thereof.

In an embodiment where the conjugate is capsaicin or a capsaicinoid, the condition can include: pain (capsaicinoids can be pain relievers) associated with shingles, arthritis, muscle soreness, sprains, strains, backaches; psoriasis; diabetes; cancer; rheumatoid arthritis; fibromyalgia; and a combination thereof. In addition, BHQ-capsaicin or BHQ-VNA can be used to study the action of capsaicinoids, induce sensory activity (e.g., pain, heat, taste) in neurons and neural circuits by activation of capsaicin receptors (e.g., TRP channels), trigger apoptosis in cancer cells, and a combination thereof.

In an embodiment where the conjugate is dopamine, the condition can include: addiction, Parkinson's disease, dystonia, schizophrenia, attention deficit hyperactivity disorder (ADHD), degenerative brain disorders, heart rate regulation, blood pressure regulation, personality disorders, reward-driven learning, and a combination thereof. In addition, BHQ-dopamine can be used to study the action of dopamine and dopaminergic signaling pathways and reward circuits in behavior, cognition, motivation, prolactin production (impacts lactation and sexual gratification), sleep, mood, attention, memory, and learning, and a combination thereof.

In an embodiment where the conjugate is epinephrine, the condition can include: cardiac arrest, anaphylaxis, bronchospasam, hypoglycemia, superficial bleeding, and a combination thereof.

In an embodiment where the conjugate is norepinephrine, the condition can include: attention deficit hyperactivity disorder, depression, schizophrenia, hypotension, Alzheimer's disease, and a combination thereof.

In an embodiment where the conjugate is morphine or oripavine, the condition can include: acute and chronic pain, acute pulmonary edema, shortness of breath, addiction, withdrawal, seizures, and a combination thereof.

In an embodiment where the conjugate is estrogen (e.g., estriol, estradiol, or estrone), the condition can include: contraception, menopause, osteoporosis, lactation suppression, cancer, prostate cancer, wound healing, bulimia nervosa, and a combination thereof.

In an embodiment where the conjugate is tyrosine or a protected tyrosine (e.g., Fmoc-protected tyrosine), the condition can include: stress, cold fatigue. In addition, BHQ-tyrosine can be used to study protein kinases, signal transduction, photosynthesis. BHQ-tyrosine can be incorporated into proteins and peptides.

In an embodiment where the conjugate is hydroxytamoxifen, the condition can include: breast cancer, McCune-Albright syndrome, infertility, gynecomastia, bipolar disorder, angiogenesis, Riedel's thyroiditis. In addition, BHQ-hydroxytamoxifen can be used as a research tool to control gene expression in genetically modified organisms.

### EXAMPLE

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Serotonin (5HT) is an important neurotransmitter in the central nervous system that regulates cognitive function, sleep, mood, and appetite. It is involved in many neurologic and psychiatric diseases. Several recent lines of evidence, including patient studies, have suggested that 5HT plays a role in epileptic seizure.¹⁻⁶ Serotonergic signaling is also important in non-neuronal cells during embryonic morphogenesis, which includes gastrulation, craniofacial and bone patterning, and the generation of left-right asymmetry.^{7,8} A photochemically activatable 5HT (i.e., caged 5HT) provides a means of studying the role of 5HT in normal and disease physiology. One that is sensitive to 2-photon excitation (2PE) would provide even greater control over the release of 5HT and hence provide more details about the action of 5HT and the physiological role of 5HT.

### Discussion:

This example discloses the design, synthesis, and photochemistry of two caged serotonins: BHQ-O-5HT and BHQ-N-5HT (Scheme 1.1). These caged serotonins have excellent sensitivity to photolysis and release of 5HT at 370 nm (1PE) and at 740 nm (2PE). This example also describes the preliminary experiments showing the photochemical release of 5HT and seizure quieting in an ex vivo zebrafish brain preparation. In the example, R₁=Br, but it can also be H, F, Cl, I, or CN, and R₂=H, but it can also be a F, Cl, Br, I, OH, OR (where R is an alkyl or aryl), NRR′ (where R is H or an alkyl or aryl and R′ is H or an alkyl or aryl), CH₃, CN, or an alkyl or aryl.

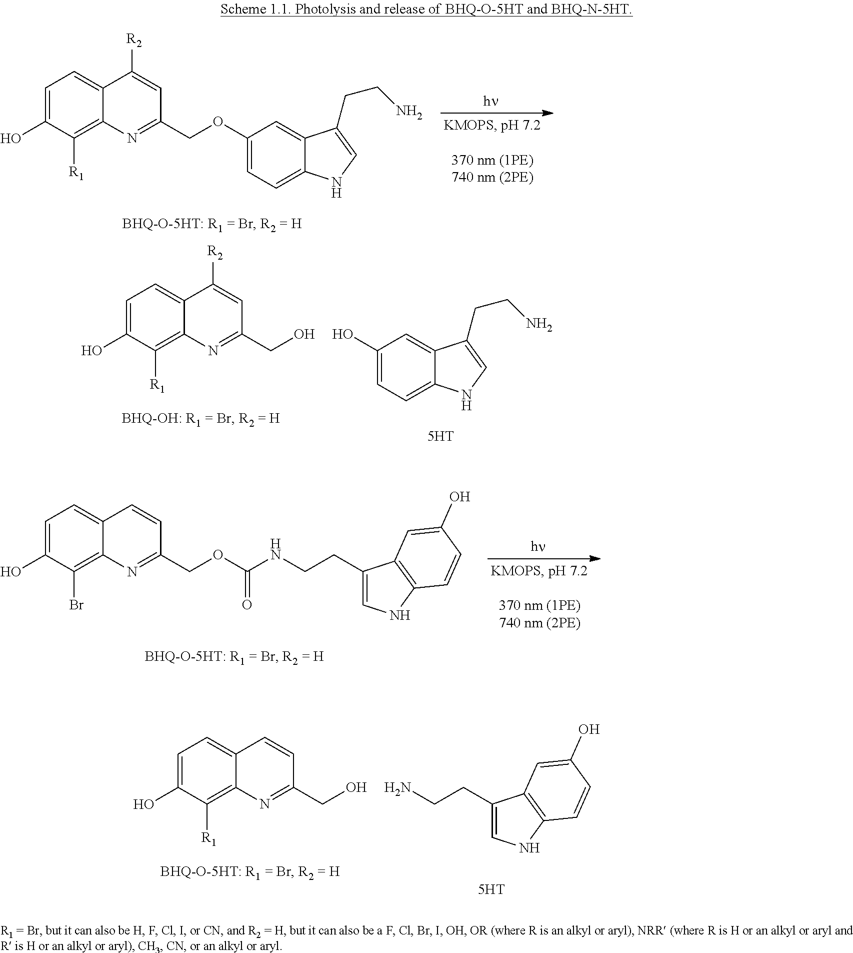

The design of BHQ-O-5HT stems from the fact that serotonin has a phenol functional group that is important for its biological activity and that blocking it with a photoremovable protecting group renders 5HT inactive. We know from our previous work (Zhu & Dore, unpublished) that BHQ can protect phenol and mediate its photochemical release by 1PE and 2PE processes (Scheme 1.2). BHQ-OPh is synthesized in 3 steps from MOM-BHQ-OH,¹¹ a known compound. BHQ-OPh absorbs light in the UV A region of the spectrum (λₘₐₓ=369, ε=3200 M⁻¹ cm⁻¹) and in neutral buffered aqueous solutions (pH 7.2 KMOPS) undergoes 1-photon photolysis at 365 nm with a quantum efficiency Qᵤ=0.19 and 2-photon photolysis uncaging action cross section δᵤ=0.56 GM at 740 nm. BHQ-OPh is stable in the dark under simulated physiological conditions: time constant for hydrolysis in the dark τ_{dark}=95 h.

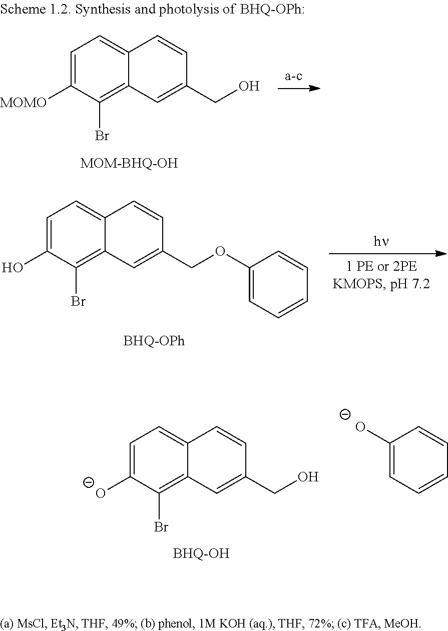

### Preparation of BHQ-O-5HT, BHQ-N-5HT, CyHQ-O-5HT, and CyHQ-N-5HT:

BHQ-O-5HT was prepared as show in Scheme 1.3. Starting from the known compound, MOM-BHQ-OH,^{11,12} the mesylate was formed, which was subsequently displaced by Boc-protected serotonin. Global deprotection with TFA revealed BHQ-O-5HT. Alternative strategies involve using different protecting groups. The MOM group can also be β-methoxyethoxymethyl ether (MEM), methyl (Me), methyl thiomethyl (MTM), benzyloxymethyl (BOM), tetrahydropyranyl (THP), ethoxyethyl (EE), trityl (Tr), methoxytrityl, benzene sulfonyl (Bs), toluenesulfonyl (Ts), or any silicon-based protecting group (e.g., TBS, TBDPS, TIPS). The mesylate (OMs) can also be I, Br, or Cl. The Boc group can also be 9-fluorenylmethyloxycarbonyl (Fmoc), benzyloxy carbonyl (Cbz or Z), or allyloxycarbonyl (alloc).

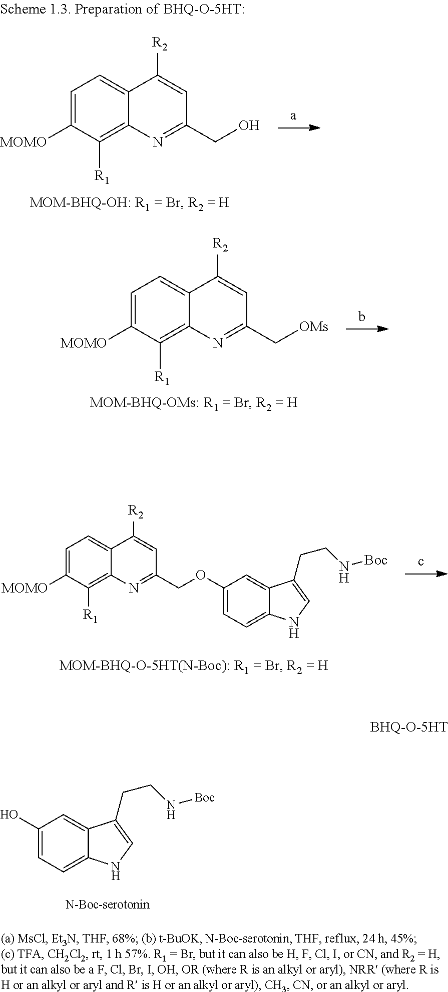

BHQ-N-5HT was prepared as shown in Scheme 1.4. The carbonyldiimidazole of MOM-BHQ was generated from MOM-BHQ-OH. Coupling to O-TIPS protected serotonin produced the protected version of BHQ-N-5HT. The TIPS group was removed first using tetrabutylammoniuum fluoride, followed by removal of the MOM group under acidic conditions to provide BHQ-N-5HT. The MOM group can also be β-methoxyethoxymethyl ether (MEM), methyl (Me), methyl thiomethyl (MTM), benzyloxymethyl (BOM), tetrahydropyranyl (THP), ethoxyethyl (EE), trityl (Tr), methoxytrityl, benzene sulfonyl (Bs), toluenesulfonyl (Ts), or any silicon-based protecting group (e.g., TBS, TBDPS, TIPS). The mesylate (OMs) can also be I, Br, or Cl. The TIPS group can also be another silicon protecting group such as TBS or TBDPS.

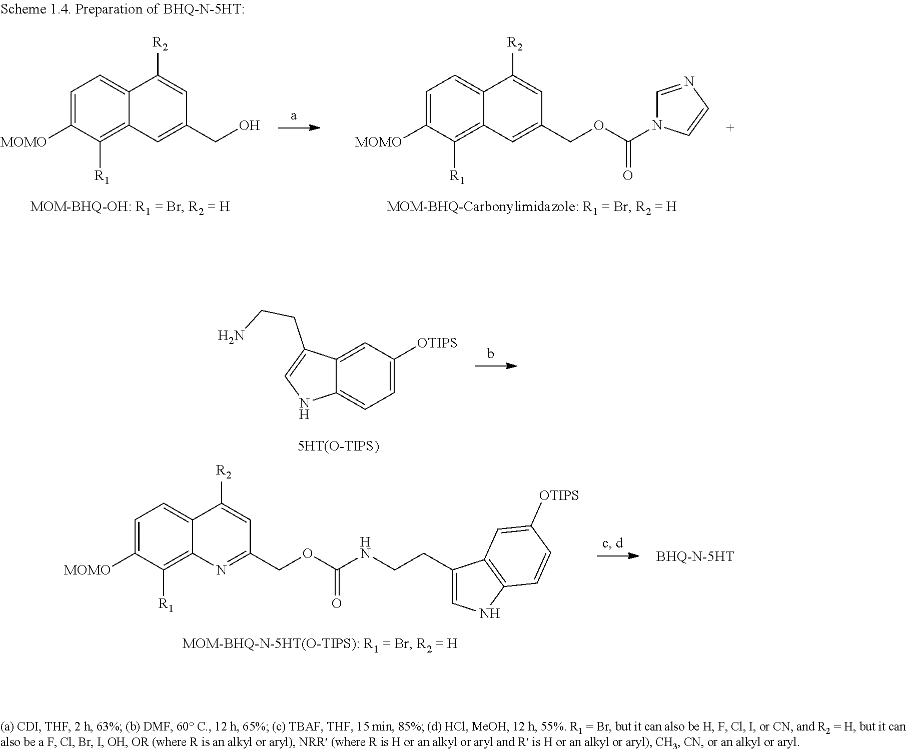

CyHQ-O-5HT and CyHQ-N-5HT (R₁=CN) can be prepared from MOM-CyHQ-OH¹³ similarly to the BHQ versions described in Schemes 1.3 and 1.4.

### Procedures for Preparing BHQ-O-5HT:

MOM-BHQ-OMs.

MOM-BHQ-OH (0.526 g, 1.76 mmol) was dissolved in THF. Methanesulfonyl chloride (0.20 mL, 2.64 mmol) and diisopropyl ethyl amine (0.61 mL, 3.52 mmol) were added dropwise and the reaction stirred at rt for 2 h. The reaction was concentrated in vacuo and the residue purified over silica gel with a gradient from 100% hexanes to 2:3 EtOAc/hexanes, collecting the product as a white solid (0.446 g, 68%): ¹H NMR (400 MHz, CDCl₃) δ 8.19 (d, 1H), 7.79 (d, 1H), 7.55 (d, 1H), 7.52 (d, 1H), 5.57 (s, 2H), 5.43 (s, 2H), 3.58 (s, 3H), 3.23 (s, 3H); ¹³C NMR (101 MHz, CDCl₃) δ 155.8, 155.7, 146.1, 137.9, 128.2, 124.9, 118.7, 118.0, 112.5; 95.6, 72.4, 56.9, 38.7; HRMS-ESI (m/z) calcd for [M+H]⁺331.9587, 333.9567. found 331.9587, 333.9566.

MOM-BHQ-O-5HT(N-Boc).

MOM-BHQ-OMs (0.071 g, 0.19 mmol) was dissolved in THF. Serotonin (N-Boc) (0.052 g, 0.19 mmol) and potassium tert-butoxide (0.031 g, 0.28 mmol) were added and the reaction stirred at reflux for 24 h. The reaction was allowed to cool, and concentrated. The residue was purified by column chromatography with 10:1 CHCl₃/acetone. Fractions were collected and concentrated (0.047 g, 45%): ¹H NMR (400 MHz, CDCl₃) δ 8.13 (d, 1H), 7.97 (s, 1H), 7.74 (t, 2H), 7.50 (d, 1H), 7.23 (s, 1H), 7.02 (s, 2H), 5.50 (s, 2H), 5.42 (s, 2H), 3.59 (s, 3H), 3.42 (t, 2H), 2.90 (t, 2H), 1.43 (s, 9H); ¹³C NMR (101 MHz, CDCl₃) δ 170.7, 161.2, 161.1, 160.6, 156.1, 155.3, 153.0, 146.0, 137.2, 131.9, 128.2, 124.7, 118.5, 117.3, 112.8, 112.1, 103.5, 102.6, 95.6, 77.4, 72.2, 56.9, 40.8, 28.7, 26.0; HRMS-ESI (m/z) calcd for [M+H]⁺ 556.1447, 558.1427. found 556.1432, 558.1420.

BHQ-O-5HT.

MOM-BHQ-O-5HT(N-Boc) (0.047 g, 0.085 mmol) was dissolved in methylene chloride. Trifluoroacetic acid was added and the reaction stirred at rt for 1 h. The solvent was removed in vacuo and the residue purified by HPLC with 50% CH₃CN/50% H₂O (w/0.1% TFA). Fractions containing only one peak were combined and concentrated (0.020 g, 57%): ¹H NMR (400 MHz, (CD₃)₂CO) δ 8.31 (d, 1H), 7.85 (d, 1H), 7.67 (d, 1H), 7.41 (d, 1H), 7.39 (s, 1H), 7.35 (s, 1H), 7.27 (d, 1H), 6.96 (d, 1H), 5.41 (s, 2H), 4.07 (t, 2H), 3.26 (t, 2H); ¹³C NMR (101 MHz, (CD₃)₂CO); 161.9, 160.2, 160.7, 156.3, 155.8, 153.4, 146.1, 138.5, 131.0, 127.4, 123.2, 119.7, 118.4, 112.2, 113.0, 102.8, 102.1, 77.6, 73.1, 40.6, 25.2; HRMS-ESI (m/z) calcd for [M+H]⁺ 412.0661, 414.0640. found 412.0651, 414.0626.

### Procedures for Preparing BHQ-N-5HT:

MOM-BHQ-Carbonylimidazole.

MOM-BHQ-OH (0.100 g, 0.34 mmol) was dissolved in THF. Carbonyldiimidazole (0.082 g, 0.50 mmol) was added, and the reaction stirred at rt for 2 h. The reaction was concentrated and the residue dissolved in EtOAc, washed with water and brine, dried over MgSO₄, filtered, and concentrated in vacuo. The crude product was puffed by column chromatography with silica gel, eluting with a gradient from 1:1 EtOAc/Hexanes to 100% EtOAc, yielding a white solid (0.084 g, 63%): ¹H NMR (400 MHz, CDCl₃) δ 8.28 (s, 1H), 8.14 (d, J=8.4 Hz, 1H), 7.74 (d, J=9.0 Hz, 1H), 7.55 (s, 1H), 7.50 (d, J=9.0 Hz, 1H), 7.39 (d, J=8.4 Hz, 1H), 5.75 (s, 2H), 5.39 (s, 2H), 3.56 (s, 3H); ¹³C NMR (101 MHz, CDCl₃) δ 155.8, 155.4, 148.9, 146.1, 137.6, 137.6, 131.0, 128.0, 124.7, 118.0, 117.7, 117.6, 112.6, 95.6, 69.9, 56.8; HRMS-ESI (m/z) calcd for [M+H]⁺392.0246, 394.0225; found 392.0262, 394.0244.

MOM-BHQ-N-5HT(O-TIPS).

O-TIPS-protected 5HT (0.067 g, 0.020 mmol) was dissolved in a small amount of DMF. MOM-BHQ-Carbonylimidazole (0.100 g, 0.25 mmol) was added and the reaction heated to 60° C. and stirred overnight. The solvent was removed in vacuo and the residue partitioned between EtOAc and water. The combined organic extracts were dried over MgSO₄, filtered, and concentrated in vacuo. The crude product was purified by column chromatography with silica gel, eluting with a gradient from 100% hexanes to 1:1 EtOAc/hexanes, yielding the product as a solid (0.0859 g, 65%): ¹H NMR (400 MHz, CDCl₃) δ 8.07 (d, J=8.4 Hz, 1H), 7.95 (s, 1H), 7.72 (d, J=9.0 Hz, 1H), 7.48 (d, J=9.0 Hz, 1H), 7.36 (d, J=8.4 Hz, 1H), 7.18 (d, J=8.7 Hz, 1H), 7.04 (s, 1H), 7.00 (s, 1H), 6.81 (d, J=8.7 Hz, 1H), 5.44 (s, 2H), 5.40 (s, 2H), 3.58 (t, J=6.6 Hz, 2H), 3.57 (s, 3H), 2.95 (t, J=6.6 Hz, 2H), 1.26 (m, J=7.3 Hz, 3H), 1.11 (d, J=7.3 Hz, 18H); ¹³C NMR (126 MHz, CDCl₃) δ 158.8, 156.2, 155.2, 149.7, 145.8, 137.0, 131.8, 127.9, 127.8, 124.4, 122.9, 118.1, 117.2, 116.3, 112.3, 111.4, 107.8, 95.4, 77.2, 67.5, 56.6, 41.2, 25.7, 18.1, 12.7; HRMS-ESI (m/z) calcd for [M+H]⁺ 656.2155, 658.2135. found 656.2171, 658.2154.

MOM-BHQ-N-5HT.

MOM-BHQ-N-5HT(O-TIPS) (85.9 mg, 0.13 mmol) was dissolved in a small amount of THF. TBAF (0.2 mL, 1.0 M in THF) was added slowly and the reaction stirred at rt for 15 min. The reaction was concentrated and the residue partitioned between EtOAc and water. The organic layer was washed with water and brine, dried over anhydrous MgSO₄, filtered, and concentrated in vacuo. The crude product was purified by column chromatography with silica gel, eluting with a gradient from 100% hexanes to 1:1 EtOAc/hexanes, yielding the product as a solid (55 mg, 85%): ¹H NMR (400 MHz, CDCl₃) δ 8.05 (d, 1H), 8.00 (s, 1H), 7.68 (d, 1H), 7.35 (d, 1H), 7.18 (d, 1H), 6.95 (m, 2H), 6.77 (d, 1H), 5.42 (s, 2H), 5.38 (s, 2H), 5.18 (broad, 1H), 3.57 (s, 3H), 3.48 (q, 2H), 2.84 (t, 2H); ¹³C NMR (101 MHz, CDCl₃) δ 158.8, 156.7, 155.5, 149.8, 145.9, 137.4, 131.8, 128.1, 126.2, 124.6, 123.4, 118.3, 117.5, 115.8, 112.3, 112.1, 103.2, 103.3, 95.6, 67.7, 56.9, 41.6, 26.0; HRMS-ESI (m/z) calcd for [M+H]⁺ 500.0821, 502.0801. found 500.0823, 502.0810.

BHQ-N-5HT.

MOM-BHQ-N-5HT (45 mg, 0.090 mmol) was dissolved in methanol. A small amount of conc. HCl was added and the reaction stirred overnight. The reaction was diluted with EtOAc and washed with sat. NaHCO₃ and brine, dried over anhydrous MgSO₄, filtered, and concentrated in vacuo. The crude product was purified by HPLC with 50% CH₃CN/50% H₂O (w/0.1% TFA) and the first peak (ret. time 4.5 min) was collected and concentrated (22.5 mg, 55%): ¹H NMR (500 MHz, (CD₃)₂CO) δ 8.11 (d, J=8.3 Hz, 1H), 7.68 (d, J=8.8 Hz, 1H), 7.26 (d, J=8.3 Hz, 2H), 7.23 (d, J=8.8 Hz, 1H), 7.07 (d, J=8.6 Hz, 1H), 6.98 (s, 1H), 6.88 (s, 1H), 6.57 (d, J=8.6 Hz, 1H), 5.22 (s, 2H), 3.34 (t, J=7.3 Hz, 2H), 2.79 (t, J=7.3 Hz, 2H); ¹³C NMR (101 MHz, (CD₃)₂CO) δ 159.1, 156.2, 155.8, 150.7, 145.9, 137.1, 131.6, 128.5, 128.1, 127.6, 123.2, 118.6, 116.8, 111.6, 111.5, 106.9, 102.6, 66.8, 41.6, 29.7, 25.9; HRMS-ESI (m/z) calcd for [M+H]⁺456.0559, 458.0538. found 456.0574, 458.0567.

### Procedures for Preparing CyHQ-O-5HT:

MOM-CyHQ-OMs.

MOM-CyHQ-OH (0.429 g, 1.76 mmol) was dissolved in THF.

Methanesulfonyl chloride (0.20 mL, 2.64 mmol) and diisopropyl ethyl amine (0.61 mL, 3.52 mmol) were added dropwise and the reaction stirred at rt for 2 h. The reaction was concentrated in vacuo and the residue purified over silica gel with a gradient from 100% hexanes to 2:3 EtOAc/hexanes, collecting the product as a white solid (0.255 g, 45%): ¹H NMR (400 MHz, CDCl₃) δ 8.23 (d, 1H), 8.05 (d, 1H), 7.59 (d, 1H), 7.53 (d, 1H), 5.57 (s, 2H), 5.43 (s, 2H), 3.58 (s, 3H), 3.23 (s, 3H); ¹³C NMR (101 MHz, CDCl₃) δ 162.5, 157.0, 148.2, 137.5, 133.7, 122.9, 119.1, 116.3, 114.5, 99.5, 95.1, 71.6, 56.9, 38.5; HRMS-ESI (m/z) calcd for [M+H]⁺324.0730. found, 324.0726.

MOM-CyHQ-O-5HT(N-Boc).

MOM-CyHQ-OMs (0.050 g, 0.155 mmol) was dissolved in THF (2 mL) and N-Boc-serotonin (0.031 g, 0.155 mmol) was added. 1M KOH (0.25 mL) was added and the reaction was stirred overnight. The reaction was concentrated in vacuo and purified by column chromatography with 1:1 EtOAc:hexane. The solvent was removed in vacuo (0.038 g, 49%) to provide MOM-CyHQ-O-5 HT (N-Boc): ¹H NMR (400 MHz, CDCl₃) δ 8.16 (d, 1H), 8.03 (s, 1H) 7.97 (d, 1H), 7.78 (d, 1H), 7.53 (d, 1H), 7.23 (s, 1H), 6.99 (m, 2H), 5.49 (s, 2H), 5.46 (s, 2H), 3.59 (s, 3H), 3.42 (t, 2H), 2.90 (t, 2H), 1.43 (s, 9H); ¹³C NMR (101 MHz, CDCl₃) δ 170.7, 162.5, 161.1, 160.6, 157.1, 155.3, 149.6, 146.0, 135.9, 133.7, 128.1, 122.9, 118.9, 117.3, 112.8, 112.1, 103.5, 102.6, 97.6, 95.6, 77.4, 72.2, 56.9, 40.8, 28.7, 26.0; HRMS-ESI (m/z) calcd for [M+H]⁺503.2289. found, 503.2295.

CyHQ-O-5HT.

MOM-CyHQ-O-5HT(N-Boc) (0.038 g, 0.075 mmol) was dissolved in methanol. Trimethylsilylchloride was titrated in to the solution until the reaction was complete by TLC. The solvent was removed in vacuo and the residue purified by HPLC with 50% CH₃CN/50% H₂O (w/0.1% TFA). Fractions containing only one peak were combined and concentrated to provide CyHQ-O-5HT: ¹H NMR (400 MHz, CD₃OD) δ 8.28 (d, 1H), 8.15 (d, 1H), 7.60 (d, 1H), 7.45 (s, 1H), 7.44 (s, 1H), 7.38 (d, 1H), 7.24 (d, 1H), 7.00 (d, 1H), 5.21 (s, 2H), 4.06 (t, 2H), 3.30 (t, 2H); ¹³C NMR (101 MHz, CD₃OD) δ 161.3, 155.7, 153.2, 145.7, 134.8, 133.7, 131.5, 127.9, 123.4, 120.5, 119.5, 117.6, 113.8, 111.2, 107.4, 104.9, 101.4, 93.8, 71.3, 48.2, 23.4; HRMS-ESI (m/z) calcd for [M+H]⁺359.1503. found, 359.1503.

### Photochemical Properties of BHQ-O-5HT and BHQ-N-5HT:

Both BHQ-O-5HT and BHQ-N-5HT exhibit excellent photochemical properties for use in vivo. Data are summarized in Table 1.1. The quantum efficiency (Qᵤ) for photolysis of BHQ-O-5HT at 365 nm, which is not detrimental to biological tissues, is similar to other BHQ-caged compounds and quite high relative to other protecting groups for biological use.^{14,15} The 2-photon uncaging action cross-section (δᵤ) a measure of the sensitivity of the compound to 2PE-mediated release of 5HT is also similar to other BHQ-caged compounds and sufficiently high for biological use.

TABLE 1.1
Photophysical and Photochemical Properties of BHQ-O-5HT
and BHQ-N-5HT

| | λₘₐₓ | ε | | Sensitivity | δᵤ | |
|---|---|---|---|---|---|---|
| Caged 5HT | nm | M⁻¹ cm⁻¹ | Qᵤ | Qᵤ × ε | GM | τ_{dark} h |
| BHQ-O-5HT | 368 | 2,000 | 0.30 | 600 | 0.50 | 260 |
| BHQ-N-5HT | 370 | 2,100 | 0.10 | 210 | 0.42 | 300 |

### Procedures for Measuring the Photochemical Properties of BHQ-O-5HT and BHQ-N-5HT:

Determination of the Molar Extinction Coefficient (ε).

A weighed portion of BHQ-O-5HT was dissolved in methanol. A measured aliquot of this solution was withdrawn and placed in KMOPS buffer (3.0 mL) and mixed thoroughly to generate a 100-μM solution of BHQ-O-5HT. The absorbance A of this solution at τₘₐₓ=368 nm was measured. This method was repeated twice with different masses of BHQ-O-5HT. The three absorbencies were averaged and the molar extinction coefficient at τₘₐₓ=368 nm was calculated to be 2,000 M⁻¹ cm⁻¹ using the equation A=ε1c, where A is the absorbance, 1 is the path length of the cuvette, and c is the concentration of the solution. The procedure was repeated for BHQ-N-Serotonin, and ε was determined to be 2,100 M⁻¹ cm⁻¹.

Determination of the Uncaging Quantum Efficiency (Qᵤ).

The quantum efficiency was calculated using the equation Qᵤ=(Iσt_{90%})⁻¹, where I is the irradiation intensity in einstein·cm⁻², σ is the decadic extinction coefficient (1,000 times ε) and t_{90%} is the time in seconds required for the conversion of 90% of the starting material to product. To find t_{90%}, a solution of BHQ-O-5HT in KMOPS was prepared and placed in a cuvette along with a small stir bar. While stirring, the solution was irradiated with UV light from a mercury lamp (Spectroline SB-100P, Spectronics Corporation) equipped with two glass filters (CS0-52, CS7-60, Ace Glass) so that the wavelength was restricted to 365±15 nm. Periodically, 20-μL aliquots were removed and analyzed by HPLC. The time points collected were as follows: 0, 5, 10, 20, 30, 60, 90, and 120 s. Percent BHQ-O-5HT remaining was plotted verses time of photolysis. A simple single exponential decay curve provided the best fit for the data and was used to extrapolate t_{90%}. The lamp's UV intensity I was measured using potassium ferrioxalate actinometry. Initially, 6 mM potassium ferrioxalate solution (3 mL) was irradiated with the mercury lamp for 60 s. A portion of this solution (2 mL) was combined with aqueous buffer (3 mL), 0.1% phenanthroline solution (3 mL), and 2M KF solution (1 mL) in a 25-mL volumetric flask. Deionized water was added to generate a 25 mL solution. A blank solution was also prepared using the same method, but the potassium ferrioxalate used in the blank was not irradiated. Both solutions were allowed to sit for one hour and the blank was then used as a baseline against which the absorbance of the irradiated solution was measured at 510 nm. The following equation was used to calculate lamp intensity:

$$I = \frac{{V}_{3} Δ {D}_{510}}{1000 {ɛ}_{510} {V}_{2} {φ}_{Fe} t}$$

where V₃ is the volume of dilution (25 mL), V₂ is the volume of irradiated potassium ferrioxalate solution taken for analysis (2 mL), ΔD₅₁₀ is the absorption of the solution at 510 nm, Σ₅₁₀ is the actinometry extinction coefficient (1.11×10⁴ M⁻¹ cm⁻¹),)_{Fe} is the quantum yield for production of ferrous ions from potassium ferrioxalate at 365 nm, and t represents the time of irradiation. The ΔD₅₁₀ value used for calculations is the average of two measurements taken before and after irradiation of BHQ-O-5HT. Compilation of the measurements yielded an uncaging quantum efficiency Qᵤ, of 0.30. The experiment was repeated for BHQ-N-5HT, and compilation of the measurements yielded an uncaging quantum efficiency Qᵤ of 0.10.

Determination of Two-photon Action Cross-Sections (δᵤ).

A portion of BHQ-O-5HT was dissolved in KMOPS buffer and the concentration of the solution was found using UV-Vis absorption in conjunction with Beer's law. Aliquots (25 μL) of this solution were placed in a microcuvette (10×1×1 mm illuminated dimensions) and irradiated with a fs-pulsed and mode-locked Ti:Sapphire laser (Chameleon Ultra II, Coherent) with 740-nm light at an average power of 300 mW. Three samples were irradiated for each of the following time periods: 0, 5, 10, 20, and 40 min. The samples were compiled and analyzed by HPLC. A solution of fluorescein at pH 9.0 was prepared to act as a standard for BHQ-O-5HT because of its well-characterized 2PE cross-section (δ_{aF}=30 GM at 740 nm) and quantum yield (Q_{F2}=0.9). UV-V is absorption spectroscopy was used to correlate absorption at 488 nm to precise concentration. Aliquots (25 μL) of fluorescein solution were placed in the microcuvette and irradiated by the laser under the same conditions used for the BHQ-O-5HT solution. The fluorescence output of the solution was measured with a radiometer before and after the BHQ-O-5HT samples were irradiated and the two values were averaged. The following equation was used to calculate the two-photon action cross-section for BHQ-O-5HT:

$${δ}_{u} = \frac{{N}_{p} φ {Q}_{F 2} {δ}_{aF} {C}_{F}}{< F ( t ) > {C}_{S}}$$

where Nₚ is the number of product molecules formed per second (determined by HPLC), φ is the collection efficiency of the detector (SED033 on an IL-1700, International Light) used to measure the fluorescence of fluorescein passing through the cuvette window and through a 535/545 nm bandpass filter at a right angle to the laser's beam, C_{F} is the concentration of fluorescein, <F(t)> is the time averaged fluorescent photon flux (photons/s) of fluorescein and C_{S} is the initial concentration of the caged compound. The measurements were compiled and the two-photon action cross-section for BHQ-O-5HT was determined to be 0.50 GM. The experiment was repeated for BHQ-N-5HT, and the two-photon action cross-section was determined to be 0.42 GM.

Determination of the Dark Hydrolysis Rate (τ_{dark}).

Three 100-μM solutions of BHQ-O-5HT in KMOPS were created and stored in the dark. Aliquots (20 μL) were removed periodically from each solution and analyzed by HPLC. The percents remaining for each time point for each solution were averaged and plotted versus time. A simple single exponential decay curve provided the best fit. The time constant for dark hydrolysis (τ_{dark}) was determined to be 260 h. The experiment was repeated for BHQ-N-5HT, and the time constant for dark hydrolysis (τ_{dark}) was determined to be 300 h.

### Biological Study of Caged 5HT in Ex Vivo Zebrafish Brain:

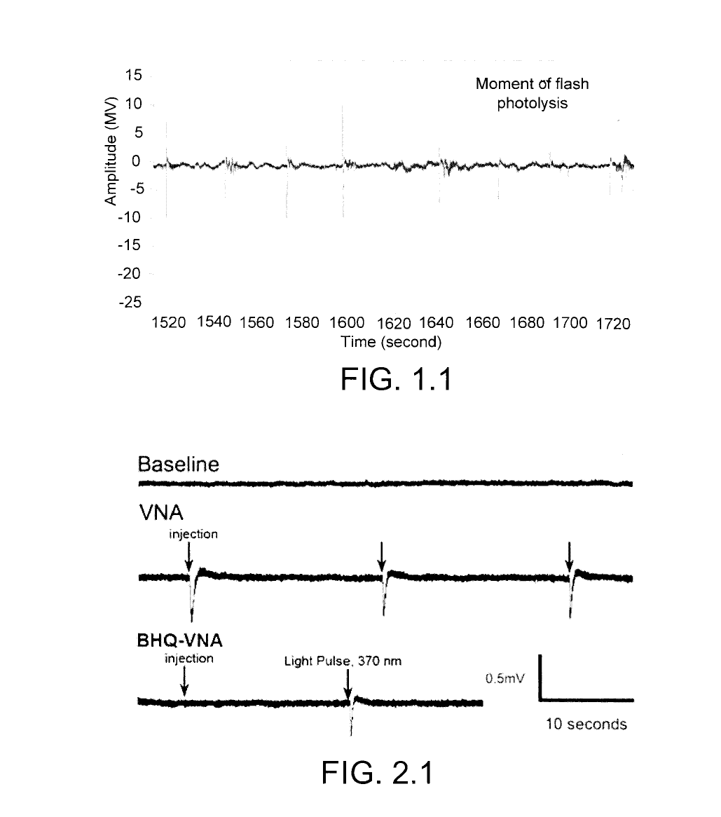

BHQ-O-5HT Mediates the Light Activation of 5HT in an Ex Vivo Zebrafish Brain Preparation (FIG. 1.1).

A microelectrode inserted into the optic tectum was used to record seizure activity induced by application of pentalenetetrazole (PTZ, 15 mM). The ictal and interictal spikes can be observed at regular intervals. After 1040 s, BHQ-O-5HT (1 mM) was added. No change in the amplitude of the ictal spikes was observed. At 1640 s, the preparation was exposed to a brief (˜1 ms) flash of 365-nm light. An immediate reduction in the amplitude of the ictal spikes was observed. Since 5HT is an inhibitor of seizures,^{1,2} this is the expected result.

Procedure for electrophysiological recording in ex vivo zebrafish brain:

An adult zebrafish brain was obtained by dissection in oxygenated artificial cerebrospinal fluid (ACSF).* A sharp glass pipet microelectrode (15 MΩ impedance), loaded with ACSF, was inserted into the optic tectum. A chloride-coated silver wire (0.010 in., A-M Systems, Inc. Sequim, Wash.) reference (15 MΩ impedance) was placed contra-laterally, adjacent to the brain. Neuronal activity was recorded using an MDA-41 AC Differential Amplifier (Bak Electronics, Mount Airy, Md.) and a Tektronix DPO 3012 Digital Phosphor Oscilloscope (Beaverton, Oreg.) using MATLAB. Baseline activity was recorded for 5 min followed by introduction of PTZ (15 mM). After 20-30 minutes, BHQ-O-5HT (0.99 mM) was added and 10 minutes after introduction, the experimental dish was subjected to flash photolysis (400 v 2000 uF) from a xenon arc lamp (OptoFlash, Cairn Research, Faversham, UK) filtered through a 365/10 nm bandpass filter (Chroma Technology, Bellows Falls, Vt.). The response was recorded for 30 min.

### REFERENCES, EACH OF WHICH IS INCORPORATED HEREIN BY REFERENCE

(1) Richerson, G. B.; Buchanan, G. F. The serotonin axis: Shared mechanisms in seizures, depression, and SUDEP. Epilepsia 2011, 52 Suppl 1, 28-38.
(2) Bagdy, G.; Kecskemeti, V.; Riba, P.; Jakus, R. Serotonin and Epilepsy. J. Neurochem. 2007, 100, 857-873.
(3) Obniska, J.; Kolaczkowski, M.; Charakchieva-Minol, S.; Nedza, K.; Dybala, M.; Bojarski, A. J. Synthesis, anticonvulsant properties and 5-HT1A/5-HT2A receptor affinity of new N-[(4-arylpiperazin-1-yl)-propyl]2-aza-spiro[4.4]nonane and [4.5]decane-1,3-dione derivatives. Pharmacol. Rep. 2005, 57, 336-344.
(4) Witkin, J. M.; Baez, M.; Yu, J.; Barton, M. E.; Shannon, H. E. Constitutive deletion of the serotonin-7 (5-HT₇) receptor decreases electrical and chemical seizure thresholds. Epilepsy Res. 2007, 75, 39-45.
(5) López-Meraz, M.-L.; González-Trujano, M.-E.; Neri-Bazán, L.; Hong, E.; Rocha, L. L. 5-HT_{1A} receptor agonists modify epileptic seizures in three experimental models in rats. Neuropharmacology 2005, 49, 367-375.
(6) Das, P.; Bell-Horner, C. L.; Machu, T. K.; Dillon, G. H. The GABA_{A} receptor antagonist picrotoxin inhibits 5-hydroxytryptamine type 3A receptors. Neuropharmacology 2003, 44, 431-438.
(7) Levin, M. Bioelectric mechanisms in regeneration: Unique aspects and future perspectives. Semin. Cell Dev. Biol. 2009, 20, 543-556.
(8) Levin, M.; Buznikov, G. A.; Lauder, J. M. Of Minds and Embryos: Left-Right Asymmetry and the Serotonergic Controls of Pre-Neural Morphogenesis. Dev. Neurosci. (Basel, Switz.) 2006, 28, 171-185. ACSF was prepared as reported in Edwards, J. G.; Michel, W. C. Pharmacological characterization of ionotropic glutamate receptors in the zebrafish olfactory bulb. Neuroscience 2003, 122, 1037-1047 (concentrations given in mM): NaCl (131), NaHCO₃ (20), KCl (2), KH₂PO₄ (1.25), MgSO₄ (2), CaCl₂ (2.5), and glucose (10) in water adjusted to pH 7.4 after equilibration for at least 1 h on ice with oxygen. The solution was sterilized through filtration and stored at 4° C.
(9) Breitinger, H.-G. A.; Wieboldt, R.; Ramesh, D.; Carpenter, B. K.; Hess, G. P. Synthesis and Characterization of Photolabile Derivatives of Serotonin for Chemical Kinetic Investigations of the Serotonin 5-HT3 Receptor. Biochemistry 2000, 39, 5500-5508.
(10) Boahen, Y. O.; MacDonald, G. M. A concise approach to caged serotonin for Fourier transform infrared (FT-IR) difference photolysis studies. J. Ghana Sci. Assoc. 2005, 7, 54-59.
(11) Zhu, Y. The Synthesis and Development of BHQ-Derived Compounds: A Probe for Dynamic Biological Studies, Doctoral Dissertation, University of Georgia, 2008.
(12) Dore, T. M.; Zhu, Y.; Reddie, K. G.; Lauderdale, J. D. BHQ-Caged Nucleotide Probes Photolysable by Two-Photon Excitation. U.S. Patent Application 12544523, Aug. 20, 2009.
(13) Davis, M. J.; Kragor, C. H.; Reddie, K. G.; Wilson, H. C.; Zhu, Y.; Dore, T. M. Substituent Effects on the Sensitivity of a Quinoline Photoremovable Protecting Group to One- and Two-Photon Excitation. J. Org. Chem. 2009, 74, 1721-1729.
(14) Dore, T. M. Multiphoton Phototriggers for Exploring Cell Physiology. In Dynamic Studies in Biology: Phototriggers, Photoswitches, and Caged Biomolecules; Goeldner, M., Givens, R. S., Eds.; Wiley-VCH: Weinheim, Germany, 2005, p 435-459.
(15) Dore, T. M.; Wilson, H. C. Chromophores for the Delivery of Bioactive Molecules with Two-Photon Excitation. In Photosensitive Molecules for Controlling Biological Function; Chambers, J. J., Kramer, R. H., Eds.; Humana Press: New York, 2011, p 57-92.

### Example 2

Capsaicin is a small molecule that is the active component in chili peppers and imparts a burning sensation by activating nociceptive sensory neurons. Activation of the receptor TRPV1 by either binding of a ligand such as capsaicin or one of its analogues, or by exposure to noxious heat (>37° C.) results in nerve terminal depolarization and generation of action potentials.¹ The TRP family of ion channels are well-understood cellular sensors that regulate response to temperature, touch, pain, and other stimuli.^{2,3} The responses observed by engineered and endogenously expressed TRPV1 channels to both applied capsaicin and exposure to heat are nearly identical,⁴ making activation of TRPV1 channels a versatile method for studying signal transduction activity of sensory neurons. A photochemically activatable TRPV1 ligand enables a deeper understanding of cellular responses to a variety of noxious stimuli. A caged TRPV1 ligand with sensitivity to 2PE adds even more spatiotemporal control over ligand release and receptor activation. The ability to engineer neurons with TRPV1 channels and selectively activate them using caged capsaicin and light is a powerful optogenetic tool for studying brain physiology.

### Discussion:

In this example we disclose the design, synthesis, and photochemistry of three caged capsaicin analogues: BHQ-Capsaicin, BHQ-VNA, and BHQ-VAA (Scheme 2.1). These caged capsaicinoids have excellent sensitivity to photolysis and release of capsaicin at 370 nm (1PE) and at 740 nm (2PE). In the example, R₁=Br, but it can also be H, F, Cl, I, or CN, and R₂=H, but it can also be a F, Cl, Br, I, OH, OR (where R is an alkyl or aryl), NRR′ (where R is H or an alkyl or aryl and R′ is H or an alkyl or aryl), CH₃, or an alkyl or aryl.

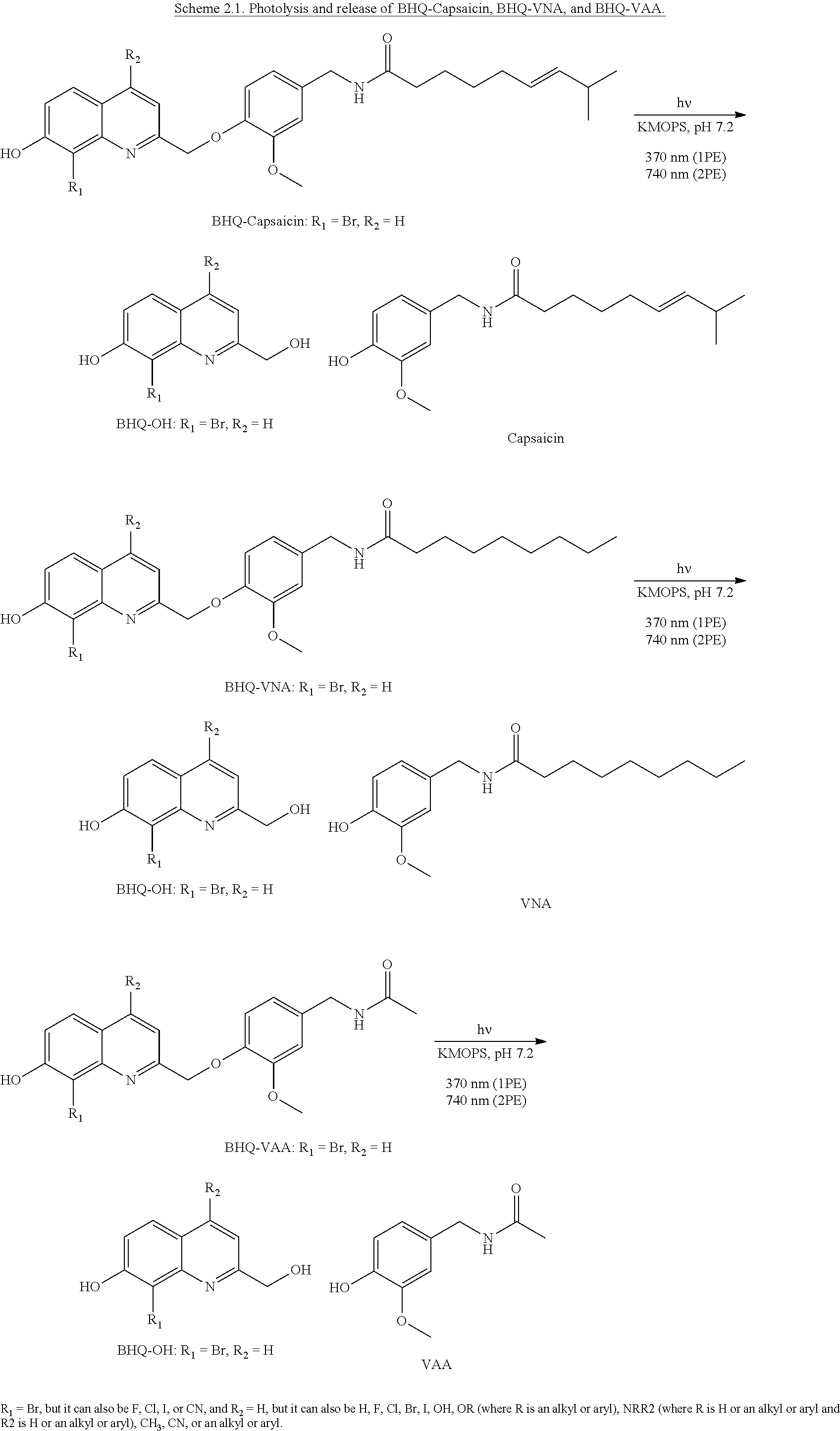

The design of BHQ-Capsaicin stems from the fact that capsaicin has a phenol functional group that is important for its biological activity and that blocking it with a photoremovable protecting group renders capsaicin inactive.¹⁰ We know from our previous work (Zhu & Dore, unpublished) that BHQ can protect phenol and mediate its photochemical release by 1PE and 2PE processes (Scheme 2.2). BHQ-OPh is synthesized in 3 steps from MOM-BHQ-OH,¹¹ a known compound. BHQ-OPh absorbs light in the UV A region of the spectrum (λₘₐₓ=369, ε=3200 M⁻¹ cm⁻¹) and in neutral buffered aqueous solutions (pH 7.2 KMOPS) undergoes 1-photon photolysis at 365 nm with a quantum efficiency Qᵤ=0.19 and 2-photon photolysis uncaging action cross section δᵤ=0.56 GM at 740 nm. BHQ-OPh is stable in the dark under simulated physiological conditions: time constant for hydrolysis in the dark Σ_{dark}=95 h.

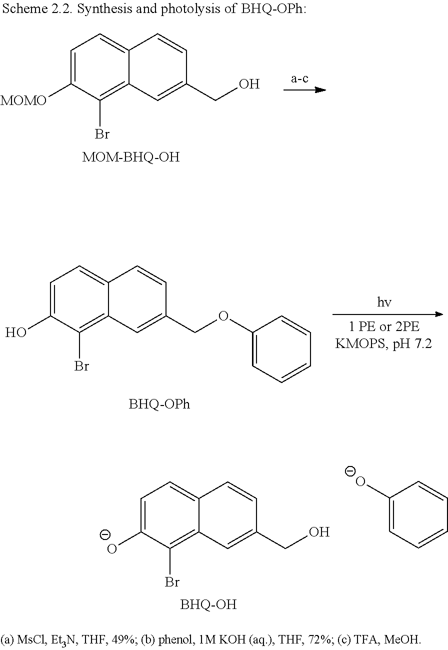

### Preparation of BHQ-Capsaicin, -VNA, and -VAA and CyHQ-Capsaicin and -VNA:

BHQ-Capsaicin, BHQ-VNA, and BHQ-VAA were prepared as shown in Scheme 2.3. Starting from the known compound, MOM-BHQ-OH, the mesylate was formed, which was subsequently displaced by capsaicin, VNA, or VAA. Global deprotection with TFA revealed BHQ-Capsaicin, BHQ-VNA, or BHQ-VAA, respectively. CyHQ-Capsaicin, -VNA, and -VAA (R₁=CN) were prepared similarly. Alternative strategies involve using different protecting groups. The MOM (methoxymethyl ether) group can also be β-methoxyethoxymethyl ether (MEM), methyl (Me), methyl thiomethyl (MTM), benzyloxymethyl (BOM), tetrahydropyranyl (THP), ethoxyethyl (EE), trityl (Tr), methoxytrityl, benzene sulfonyl (Bs), toluenesulfonyl (Ts), or any silicon-based protecting group (e.g., TBS, TBDPS, TIPS). The mesylate (OMs) can also be I, Br, or Cl.

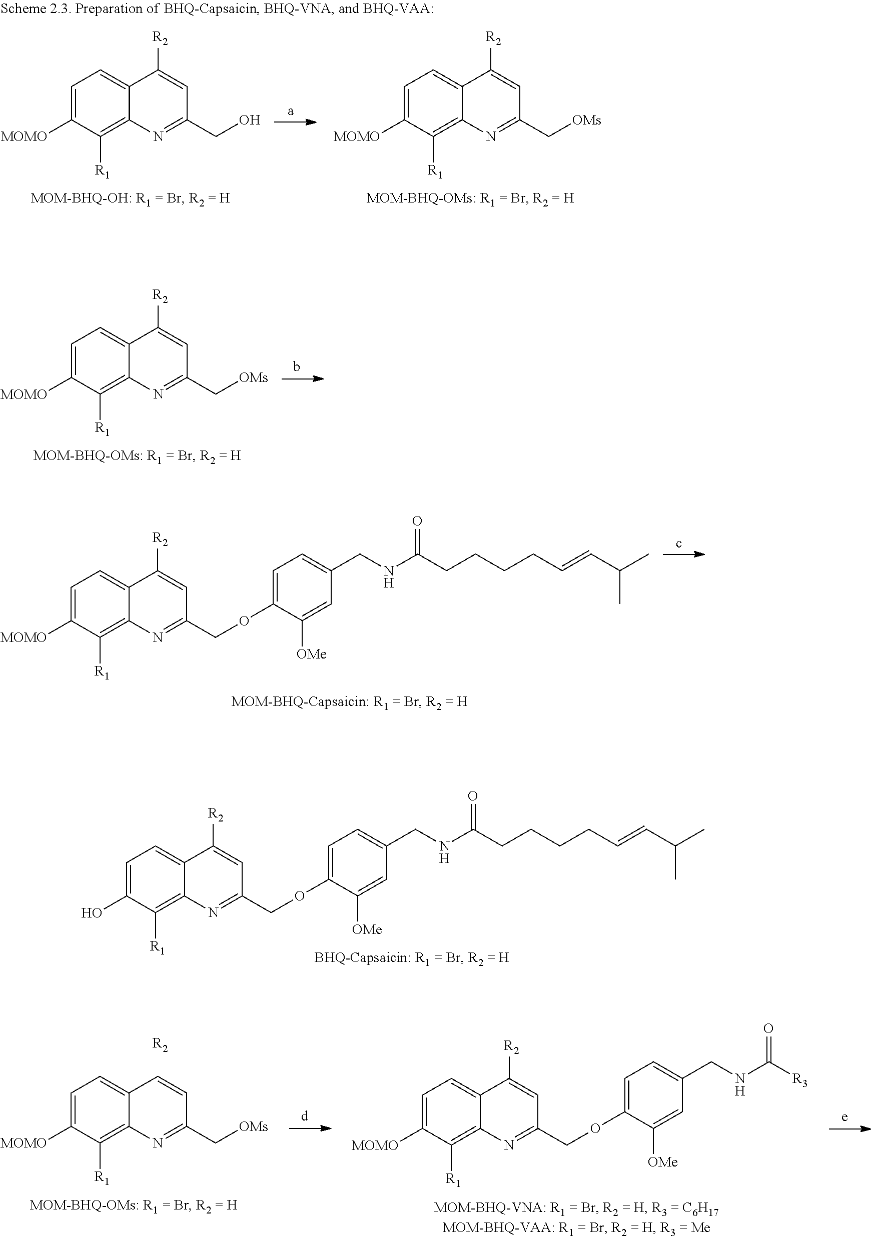

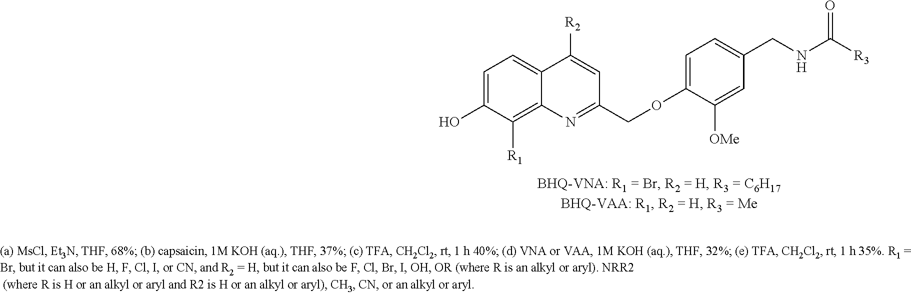

### Procedures for Preparing BHQ-Capsaicin, -VNA, and -VAA and CyHQ-Capsaicin and -VNA:

MOM-BHQ-OMs. MOM-BHQ-OH (0.526 g, 1.76 mmol) was dissolved in THF. Methanesulfonyl chloride (0.20 mL, 2.64 mmol) and diisopropyl ethyl amine (0.61 mL, 3.52 mmol) were added dropwise and the reaction stirred at rt for 2 h. The reaction was concentrated in vacuo and the residue purified over silica gel with a gradient from 100% hexanes to 2:3 EtOAc/hexanes, collecting the product as a white solid (0.446 g, 68%): ¹H NMR (400 MHz, CDCl₃) δ 8.19 (d, 1H), 7.79 (d, 1H), 7.55 (d, 1H), 7.52 (d, 1H), 5.57 (s, 2H), 5.43 (s, 2H), 3.58 (s, 3H), 3.23 (s, 3H); ¹³C NMR (101 MHz, CDCl₃) δ 155.8, 155.7, 146.1, 137.9, 128.2, 124.9, 118.7, 118.0, 112.5; 95.6, 72.4, 56.9, 38.7; HRMS-ESI (m/z) calcd for [M+H]⁺331.9587, 333.9567. found 331.9587, 333.9566.

MOM-BHQ-Capsaicin.

MOM-BHQ-OMs (0.092 g, 0.25 mmol) was dissolved in THF. Capsaicin (0.076 g, 0.25 mmol) and 1 M KOH (0.35 mL) were added and the reaction stirred overnight. The mixture was concentrated and the residue dissolved in CHCl₃. The solution washed with water and brine, dried over MgSO₄, filtered, and concentrated in vacuo. The crude product was obtained as a mixture of isomers and purified by column chromatography with 2:3 EtOAc/Hex. The solvent was removed in vacuo to provide MOM-BHQ-Capsaicin (0.054 g, 37%): ¹H NMR (400 MHz, CDCl₃) δ 8.18 (d, 1H), 7.75 (d, 1H), 7.63 (d, 1H), 7.48 (d, 1H), 6.88 (d, 1H), 6.85 (s, 1H), 6.69 (d, 1H), 5.78 (broad, 1H), 5.49 (s, 2H), 5.40 (s, 2H), 5.31 (m, 1H), 4.36 (s, 2H), 3.90 (s, 3H), 3.54 (s, 3H), 2.19 (t, 2H), 1.60 (m, 2H), 1.4-1.2 (m, 8H), 0.98 (d, 3H), 0.84 (d, 3H); ¹³C NMR (101 MHz, CDCl₃) δ 173.0, 159.9, 155.4, 149.9, 147.5, 146.0, 138.3, 137.3, 132.1, 128.1, 126.7, 124.7, 120.3, 118.2, 117.4, 114.1, 112.0, 95.7, 72.4, 56.8, 56.3, 43.6, 36.9, 32.4, 31.1, 29.5, 25.5, 22.9; HRMS-ESI (m/z) calcd for [M+H]⁺585.1964, 587.1944. found 585.1979, 587.1950

BHQ-Capsaicin.

MOM-BHQ-Capsaicin (0.054 g, 0.092 mmol) was dissolved in methylene chloride. Trifluoroacetic acid was added and the reaction stirred at rt for 1 h. The solvent was removed in vacuo and the residue purified by HPLC with 50% CH₃CN/50% H₂O (w/0.1% TFA) to separate isomers. Fractions containing only one peak corresponding to BHQ-Capsaicin were combined and concentrated to provide BHQ-Capsaicin (0.015 g, 30%): ¹H NMR (400 MHz, CDCl₃) δ 8.07 (d, J=8.4 Hz, 1H), 7.66 (d, J=8.9 Hz, 1H), 7.59 (d, J=8.4 Hz, 1H), 7.31 (d, J=8.9 Hz, 1H), 6.88 (d, J=8.2 Hz, 1H), 6.85 (s, 1H), 6.71 (d, J=8.2 Hz, 1H), 5.48 (s, 2H), 5.33 (m, 2H), 4.36 (d, 2H), 3.91 (s, 3H), 2.20 (t, J=7.6 Hz, 2H), 1.97 (q, J=6.9 Hz, 2H), 1.63 (t, J=7.6 Hz, 2H), 1.39 (t, J=7.6 Hz, 2H), 1.25 (m, broad, 1H), 0.94 (d, 6H); ¹³C NMR (101 MHz, CDCl₃) δ 173.2, 159.6, 154.6, 149.9, 147.5, 145.5, 138.3, 137.4, 132.0, 128.4, 126.6, 123.8, 120.3, 118.1, 117.6, 114.1, 112.0, 107.8, 72.4, 56.3, 43.6, 36.9, 32.4, 31.2, 29.5, 25.5, 22.9; HRMS-ESI (m/z) calcd for [M+H]⁺541.1702, 543.1681. found 541.1699, 543.1688

MOM-BHQ-VNA.

MOM-BHQ-OMs (0.107 g, 0.30 mmol) was dissolved in THF. N-vanillyl nonanamide (0.095 g, 0.32 mmol) and 1 M KOH (0.40 mL) were added and the reaction stirred overnight. The mixture was concentrated and the residue dissolved in CHCl₃. The solution washed with water and brine, dried over MgSO₄, filtered, and concentrated in vacuo. The crude product was purified by column chromatography with 2:3 EtOAc/Hex. The solvent was removed in vacuo to provide MOM-BHQ-VNA (0.055 g, 32%): ¹H NMR (400 MHz, CDCl₃) 6 (d, J=8.3 Hz, 1H), 7.75 (d, J=9.0 Hz, 1H), 7.63 (d, J=8.3 Hz, 1H), 7.48 (d, J=9.0 Hz, 1H), 6.88 (d, J=8.2 Hz, 1H), 6.85 (s, 1H), 6.69 (d, J=8.2 Hz, 1H), 5.78 (broad, 1H), 5.49 (s, 2H), 5.40 (s, 2H), 4.36 (s, 2H), 3.90 (s, 3H), 3.54 (s, 3H), 2.19 (t, J=7.6 Hz, 2H), 1.60 (m, J=7.6 Hz, 2H), 1.4-1.2 (m, 10H), 0.84 (t, J=6.7 Hz, 3H); ¹³C NMR (101 MHz, CDCl₃) δ 173.06, 159.93, 155.43, 149.88, 147.53, 146.04, 137.31, 132.09, 128.09, 124.72, 120.30, 118.22, 117.44, 114.14, 112.02, 95.68, 72.46, 56.84, 56.28, 43.59, 37.06, 32.00, 29.52, 29.49, 29.33, 25.98, 22.82, 14.25; HRMS-ESI (m/z) calcd for [M+H]⁺573.1964, 575.1944; found 573.1972, 575.1952.

BHQ-VNA.

MOM-BHQ-VNA (0.055 g, 0.096 mmol) was dissolved in methylene chloride. Trifluoroacetic acid was added and the reaction stirred at rt for 2 h. The solvent was removed in vacuo and the residue purified by HPLC with 50% CH₃CN/50% H₂O (w/0.1% TFA). Fractions containing only one peak were combined and concentrated to provide BHQ-VNA (0.029 g, 57%): ¹H NMR (400 MHz, CDCl₃) δ 8.10 (d, J=8.4 Hz, 1H), 7.70 (d, J=9.0 Hz, 1H), 7.62 (d, J=8.4 Hz, 1H), 7.33 (d, J=9.0 Hz, 1H), 6.90 (d, J=8.2 Hz, 1H), 6.87 (s, 1H), 6.73 (d, J=8.2 Hz, 1H), 5.78 (broad, 1H), 5.50 (s, 2H), 4.37 (s, 2H), 3.93 (s, 3H), 2.20 (t, J=7.6 Hz, 2H), 1.64 (m, J=7.6 Hz, 2H), 1.4-1.2 (m, 10H), 0.87 (t, J=6.7 Hz, 3H); ¹³C NMR (101 MHz, CDCl₃) δ 173.2, 159.4, 154.4, 149.6, 147.3, 145.1, 137.4, 131.7, 128.3, 123.7, 120.1, 117.9, 117.4, 114.4, 113.8, 111.7, 72.0, 56.1, 43.5, 36.8, 31.8, 29.7, 29.3, 29.1, 25.8, 22.6, 14.1; HRMS-ESI (m/z) calcd for [M+H]⁺529.1702, 531.1681. found 529.1699, 531.1689.

MOM-BHQ-VAA.

MOM-BHQ-OMs (0.073 g, 0.195 mmol) was dissolved in THF. N-vanillyl acetamide (0.038 g, 0.195 mmol) and 1 M KOH (0.3 mL) were added and the reaction stirred overnight. The mixture was concentrated and the residue dissolved in CHCl₃. The solution washed with water and brine, dried over MgSO₄, filtered, and concentrated in vacuo. The crude product was purified by column chromatography with 2:3 EtOAc/Hex. The solvent was removed in vacuo to provide MOM-BHQ-VAA (0.0312 g, 33%): ¹H NMR (400 MHz, CDCl₃) δ 8.11 (d, J=8.4 Hz, 1H), 7.74 (d, J=9.0 Hz, 1H), 7.65 (d, J=8.4 Hz, 1H), 7.50 (d, J=9.0 Hz, 1H), 6.90 (d, J=8.2 Hz, 1H), 6.87 (s, 1H), 6.73 (d, J=8.2 Hz, 1H), 5.52 (s, 2H), 5.42 (s, 2H), 4.34 (s, 2H), 3.93 (s, 3H), 3.59 (s, 3H), 2.01 (s, 3H); ¹³C NMR (101 MHz, CDCl₃)™ 169.9, 159.9, 155.5, 149.9, 147.6, 146.1, 137.3, 131.9, 128.1, 125.7, 124.7, 120.4, 118.2, 117.5, 114.2, 112.1, 95.7, 72.5, 56.8, 56.3, 43.8, 30.6; HRMS-ESI (m/z) calcd for [M+H]⁺475.0869, 477.0848. found 475.0863, 477.0852.

BHQ-VAA.

MOM-BHQ-VAA (0.0312 g, 0.066 mmol) was dissolved in methylene chloride. Trifluoroacetic acid was added and the reaction stirred at rt for 2 h. The solvent was removed in vacuo and the residue purified by HPLC with 50% CH₃CN/50% H₂O (w/0.1% TFA). Fractions containing only one peak were combined and concentrated (0.020 g, 70%): ¹H NMR (400 MHz, CD₃OD) δ 8.38 (d, J=8.4 Hz, 1H), 7.85 (d, J=8.9 Hz, 1H), 7.67 (d, J=8.4 Hz, 1H), 7.33 (d, J=8.9 Hz, 1H), 7.02 (d, J=8.2 Hz, 1H), 6.97 (s, 1H), 6.80 (d, J=8.2 Hz, 1H), 5.49 (s, 2H), 4.27 (s, 2H), 3.88 (s, 3H), 1.96 (s, 3H); ¹³C NMR (101 MHz, CD₃OD) δ 171.9, 165.6, 158.9, 150.1, 147.2, 136.4, 135.6, 133.1, 128.5, 123.7, 120.0, 119.1, 117.1, 117.1, 115.0, 112.1, 71.3, 55.4, 42.8, 21.4; HRMS-ESI (m/z) calcd for [M+H]⁺431.0606, 433.0586; found 431.0624, 433.0606.

MOM-CyHQ-OMs.

MOM-CyHQ-OH (0.429 g, 1.76 mmol) was dissolved in THF. Methanesulfonyl chloride (0.20 mL, 2.64 mmol) and diisopropyl ethyl amine (0.61 mL, 3.52 mmol) were added dropwise and the reaction stirred at rt for 2 h. The reaction was concentrated in vacuo and the residue purified over silica gel with a gradient from 100% hexanes to 2:3 EtOAc/hexanes, collecting the product as a white solid (0.255 g, 45%): ¹H NMR (400 MHz, CDCl₃) δ 8.23 (d, 1H), 8.05 (d, 1H), 7.59 (d, 1H), 7.53 (d, 1H), 5.57 (s, 2H), 5.43 (s, 2H), 3.58 (5, 3H), 3.23 (s, 3H); ¹³C NMR (101 MHz, CDCl₃) δ 162.5, 157.0, 148.2, 137.5, 133.7, 122.9, 119.1, 116.3, 114.5, 99.5, 95.1, 71.6, 56.9, 38.5; HRMS-ESI (m/z) calcd for [M+H]⁺324.0730. found 324.0726.

MOM-CyHQ-Capsaicin.

MOM-CyHQ-OMs (0.092 g, 0.25 mmol) was dissolved in THF. Capsaicin (0.076 g, 0.25 mmol) and 1 M KOH (0.35 mL) were added and the reaction stirred overnight. The mixture was concentrated and the residue dissolved in CHCl₃. The solution washed with water and brine, dried over MgSO₄, filtered, and concentrated in vacuo. The crude product was obtained as a mixture of isomers and purified by column chromatography with 2:3 EtOAc/Hex. The solvent was removed in vacuo to provide MOM-CyHQ-Capsaicin (0.054 g, 37%): ¹H NMR (400 MHz, CDCl₃) δ 8.14 (d, 1H), 7.97 (d, 1H), 7.71 (d, 1H), 7.54 (d, 1H), 6.88 (d, 2H), 6.72 (d, 1H), 5.74 (broad, 1H), 5.49 (s, 2H), 5.46 (s, 2H), 5.33 (m, 1H), 4.36 (s, 2H), 3.92 (s, 3H), 3.59 (s, 3H), 2.20 (t, 2H), 1.65 (m, 2H), 1.4-1.2 (m, 8H), 0.94 (d, 3H), 0.85 (d, 3H); ¹³C NMR (101 MHz, CDCl₃) δ 172.3, 164.2, 160.5, 149.7, 148.3, 146.9, 137.7, 137.2, 133.9, 132.6, 126.8, 124.7, 121.7, 120.0, 117.7, 114.1, 111.8, 95.6 93.8, 71.7, 55.1, 48.2, 42.4, 38.7, 35.7, 31.8, 29.3, 25.7, 25.2; HRMS-ESI (m/z) calcd for [M+H]⁺532.2806. found 532.2806.

CyHQ-Capsaicin.

MOM-CyHQ-Capsaicin (0.054 g, 0.101 mmol) was dissolved in methylene chloride. Trifluoroacetic acid was added and the reaction stirred at rt for 1 h. The solvent was removed in vacuo and the residue purified by HPLC with 50% CH₃CN/50% H₂O (w/0.1 TFA) to separate isomers. Fractions containing only one peak corresponding to CyHQ-Capsaicin were combined and concentrated to provide CyHQ-Capsaicin (0.08 g, 17%): ¹H NMR (400 MHz, CD₃OD) δ 8.34 (d, 1H), 8.07 (d, 1H), 7.70 (d, 1H), 7.45 (d, 1H), 7.03 (d, 1H), 6.98 (s, 1H), 6.80 (d, 1H), 5.34 (d, 3H), 4.36 (d, 2H), 3.85 (s, 3H), 1.96 (t, 2H), 1.63 (m, 2H), 1.4-1.2 (m, 8H), 0.92 (d, 3H), 0.84 (d, 3H); ¹³C NMR (101 MHz, CD₃OD) δ 172.9, 164.2, 162.8, 169.6, 147.8, 146.1, 139.6, 134.6, 133.4, 130.8, 129.4, 122.5, 119.7, 118.3, 115.7, 114.3, 112.2, 109.2, 90.8, 78.2, 56.1, 43.9, 36.5, 33.2, 31.7, 28.9, 27.3, 22.8, 22.1; HRMS-ESI (m/z) calcd for [M+H]⁺ 488.2544. found 488.2544.

MOM-CyHQ-VNA.

MOM-CyHQ-OMs (0.107 g, 0.30 mmol) was dissolved in THF. N-vanillyl nonanamide (0.095 g, 0.32 mmol) and 1 M KOH (0.40 mL) were added and the reaction stirred overnight. The mixture was concentrated and the residue dissolved in CHCl₃. The solution washed with water and brine, dried over MgSO₄, filtered, and concentrated in vacuo. The crude product was purified by column chromatography with 2:3 EtOAc/Hex. The solvent was removed in vacuo to provide MOM-CyHQ-VNA (0.055 g, 32%): ¹H NMR (400 MHz, CDCl₃) δ 8.14 (d, 1H), 7.97 (d, 1H), 7.71 (d, 1H), 7.54 (d, 1H), 6.88 (m, 2H), 6.72 (m, 1H), 5.78 (broad, 1H), 5.46 (s, 2H), 5.50 (s, 2H), 4.36 (d, 2H), 3.90 (s, 3H), 3.59 (s, 3H), 2.20 (t, 2H), 1.60 (m, 2H), 1.4-1.2 (m, 10H), 0.84 (t, 3H); ¹³C NMR (101 MHz, CDCl₃) δ 172.9, 162.2, 161.3, 149.6, 148.2, 147.1, 137.0, 133.7, 130.2, 123.9, 122.8, 118.8, 115.6, 114.8, 113.9, 99.5, 95.68 71.9, 56.9, 56.1, 43.4, 38.7, 31.8, 29.3, 29.1, 25.8, 25.6, 22.6, 14.1; HRMS-ESI (m/z) calcd for [M+H]⁺520.2806. found 520.2806.

CyHQ-VNA.

MOM-CyHQ-VNA (0.055 g, 0.096 mmol) was dissolved in methylene chloride. Trifluoroacetic acid was added and the reaction stirred at rt for 2 h. The solvent was removed in vacuo and the residue purified by HPLC with 50% CH₃CN/50% H₂O (w/0.1% TFA). Fractions containing only one peak were combined and concentrated to provide CyHQ-VNA (0.029 g, 57%).: ¹H NMR (400 MHz, CD₃OD) δ 8.25 (d, 1H), 8.00 (d, 1H), 7.65 (d, 1H), 7.25 (d, 1H), 6.96 (m, 2H), 6.77 (d, 1H), 5.36 (s, 2H), 4.28 (m, 2H), 3.88 (s, 3H), 2.19 (t, 2H), 1.60 (m, 2H), 1.4-1.2 (m, 10H), 0.84 (t, 3H); ¹³C NMR (101 MHz, CD₃OD) δ 173.23, 159.6, 158.7, 147.1, 145.9, 144.5, 135.3, 134.4, 131.1, 129.4, 123.9, 120.2, 117.5, 116.2, 112.9, 112.2, 96.7, 78.5, 54.4, 40.7, 36.8, 34.2, 29.6, 28.4, 27.5, 26.8, 22.7, 20.1; HRMS-ESI (m/z) calcd for [M+H]⁺476.2544. found 476.2543.

### Photochemical Properties of BHQ-VAA:

Because of the poor solubility at concentrations high enough for HPLC analysis of BHQ-Capsaicin and BHQ-VNA in aqueous buffers, BHQ-VAA was used to evaluate the photochemical properties of the caged capsaicin analogs. Data are summarized in Table 2.1. The quantum efficiency (Qᵤ) for photolysis of BHQ-VAA at 365 nm, which is not detrimental to biological tissues, is similar to other BHQ-caged compounds and quite high relative to other protecting groups for biological use.^{11,12} The 2-photon uncaging action cross-section (δᵤ), a measure of the sensitivity of the compound to 2PE-mediated release of VAA is also similar to other BHQ-caged compounds and sufficiently high for biological use.

TABLE 2.1
Photophysical and Photochemical Properties of BHQ-VAA

| Caged | λₘₐₓ | ε | | Sensitivity | δᵤ | |
|---|---|---|---|---|---|---|
| Compound | nm | M⁻¹ cm⁻¹ | Qᵤ | Qᵤ × ε | GM | τ_{dark} h |
| BHQ-VAA | 370 | 2700 | 0.18 | 486 | 0.61 | 140 |

### Procedures for Measuring the Photochemical Properties of BHQ-VAA:

Determination of the Molar Extinction Coefficient (s).

A weighed portion of BHQ-VAA was dissolved in methanol. A measured aliquot of this solution was withdrawn and placed in KMOPS buffer (3.0 mL) and mixed thoroughly to generate a 100-μM solution of BHQ-VAA. The absorbance A of this solution at λₘₐₓ=368 nm was measured. This method was repeated twice with different masses of BHQ-VAA. The three absorbencies were averaged and the molar extinction coefficient at λₘₐₓ=368 nm was calculated to be 2700 M⁻¹ cm⁻¹ using the equation A=ε1c, where A is the absorbance, 1 is the path length of the cuvette, and c is the concentration of the solution.

Determination of the Uncaging Quantum Efficiency (Qᵤ).

The quantum efficiency was calculated using the equation Qᵤ=(Iσt_{90%})⁻¹, where I is the irradiation intensity in einstein·cm⁻², σ is the decadic extinction coefficient (1,000 times c) and t_{90%} is the time in seconds required for the conversion of 90% of the starting material to product. To find t_{90%}, a solution of BHQ-VAA in KMOPS was prepared and placed in a cuvette along with a small stir bar. While stirring, the solution was irradiated with UV light from a mercury lamp (Spectroline SB-100P, Spectronics Corporation) equipped with two glass filters (CS0-52, CS7-60, Ace Glass) so that the wavelength was restricted to 365±15 nm. Periodically, 20-μL aliquots were removed and analyzed by HPLC. The time points collected were as follows: 0, 5, 10, 20, 30, 60, 90, and 120 s. Percent BHQ-VAA remaining was plotted verses time of photolysis. A simple single exponential decay curve provided the best fit for the data and was used to extrapolate t_{90%}. The lamp's UV intensity I was measured using potassium ferrioxalate actinometry. Initially, 6 mM potassium ferrioxalate solution (3 mL) was irradiated with the mercury lamp for 60 s. A portion of this solution (2 mL) was combined with aqueous buffer (3 mL), 0.1% phenanthroline solution (3 mL), and 2M KF solution (1 mL) in a 25-mL volumetric flask. Deionized water was added to generate a 25 mL solution. A blank solution was also prepared using the same method, but the potassium ferrioxalate used in the blank was not irradiated. Both solutions were allowed to sit for one hour and the blank was then used as a baseline against which the absorbance of the irradiated solution was measured at 510 nm. The following equation was used to calculate lamp intensity:

$$I = \frac{{V}_{3} Δ {D}_{510}}{1000 {ɛ}_{510} {V}_{2} {φ}_{Fe} t}$$

where V₃ is the volume of dilution (25 mL), V₂ is the volume of irradiated potassium ferrioxalate solution taken for analysis (2 mL), ΔD₅₁₀ is the absorption of the solution at 510 nm, Σ₅₁₀ is the actinometry extinction coefficient (1.11·10⁴ M⁻¹ cm⁻¹),)_{Fe} is the quantum yield for production of ferrous ions from potassium ferrioxalate at 365 nm, and t represents the time of irradiation. The \hat{X}₅₁₀ value used for calculations is the average of two measurements taken before and after irradiation of BHQ-VAA. Compilation of the measurements yielded an uncaging quantum efficiency Qᵤ of 0.18.

Determination of Two photon Action Cross-Sections (δᵤ).

A portion of BHQ-VAA was dissolved in KMOPS buffer and the concentration of the solution was found using UV-Vis absorption in conjunction with Beer's law. Aliquots (25 μL) of this solution were placed in a microcuvette (10×1×1 mm illuminated dimensions) and irradiated with a fs-pulsed and mode-locked Ti: Sapphire laser (Chameleon Ultra II, Coherent) with 740-nm light at an average power of 300 mW. Three samples were irradiated for each of the following time periods: 0, 5, 10, 20, and 40 min. The samples were compiled and analyzed by HPLC. A solution of fluorescein at pH 9.0 was prepared to act as a standard for BHQ-VAA because of its well-characterized 2PE cross-section (δ_{aF}=30 GM at 740 nm) and quantum yield (Q_{F2}=0.9). UV-V is absorption spectroscopy was used to correlate absorption at 488 nm to precise concentration. Aliquots (25 μL) of fluorescein solution were placed in the microcuvette and irradiated by the laser under the same conditions used for the BHQ-VAA solution. The fluorescence output of the solution was measured with a radiometer before and after the BHQ-VAA samples were irradiated and the two values were averaged. The following equation was used to calculate the two-photon action cross-section for BHQ-VAA:

$${δ}_{u} = \frac{{N}_{p} φ {Q}_{F 2} {δ}_{aF} {C}_{F}}{< F ( t ) > {C}_{S}}$$

where Nₚ is the number of product molecules formed per second (determined by HPLC), φ is the collection efficiency of the detector (SED033 on an IL-1700, International Light) used to measure the fluorescence of fluorescein passing through the cuvette window and through a 535/545 nm bandpass filter at a right angle to the laser's beam, C_{F} is the concentration of fluorescein, <F(t)> is the time averaged fluorescent photon flux (photons/s) of fluorescein and C_{S} is the initial concentration of the caged compound. The measurements were compiled and the two-photon action cross-section for BHQ-VAA was determined to be 0.61 GM.

Determination of the Dark Hydrolysis Rate (τ_{dark}).

Three 100-μM solutions of BHQ-VAA in KMOPS were created and stored in the dark. Aliquots (20 μL) were removed periodically from each solution and analyzed by HPLC. The percents remaining for each time point for each solution were averaged and plotted versus time. A simple single exponential decay curve provided the best fit. The time constant for dark hydrolysis (τ_{dark}) was determined to be 140 h.

BHQ-VNA Mediates the Light Activation of VNA in Dorsal Root Ganglia Cells in Culture (FIG. 2.1):

Extracellular recordings from cultured dorsal root ganglia (DRG) prepared from an adult mouse demonstrated that topical application of VNA evoked a change in the extracellular potential. For these experiments, recordings were made from single neurons. In the absence of VNA, normal electric potential is observed (FIG. 2.1, “baseline” trace). The expected change in potential is observed immediately upon application of VNA onto a single DRG neuron in the vicinity of the electrode. VNA is a capsaicin analog that is an agonist for TRPV1 channels, which are present on the surface of dorsal root ganglia. VNA evokes a strong and immediate electrophysiological response from the cells through activation of the TRPV1 channels (FIG. 2.1, “VNA” trace). Bath applied BHQ-VNA does not have any affect on the dorsal root ganglia (FIG. 2.1, “BHQ-VNA” trace), but when a short pulse of 370-nm light is directed at the culture, an immediate potential change is observed that is indistinguishable from the VNA trace.

### REFERENCES, EACH OF WHICH IS INCORPORATED HEREIN BY REFERENCE

(1) Caterina, M. J.; Schumacher, M. A.; Tominaga, M.; Rosen, T. A.; Levine, J. D.; Julius, D. The capsaicin receptor: a heat-activated ion channel in the pain pathway. Nature 1997, 389, 816-824.
(2) Clapham, D. E.; Runnels, L. W.; Strubing, C. The TRP ion channel family. Nat. Rev. Neurosci. 2001, 2, 387-396.
(3) Clapham, D. E. TRP channels as cellular sensors. Nature 2003, 426, 517-524.
(4) Karai, L. J.; Russell, J. T.; Iadarola, M. J.; Olah, Z. Vanilloid Receptor 1 regulates multiple calcium compartments and contributes to Ca2+-induced Ca2+ release in sensory neurons. J. Biol. Chem. 2004, 279, 16377-16387.
(5) Zemelman, B. V.; Nesnas, N.; Lee, G. A.; Miesenböck, G. Photochemical gating of heterologous ion channels: Remote control over genetically designated populations of neurons. Proc. Natl. Acad. Sci. U.S.A. 2003, 1100, 1352-1357.
(6) Carr, J. L.; Wease, K. N.; Van Ryssen, M. P.; Paterson, S.; Agate, B.; Gallagher, K. A.; Brown, C. T. A.; Scott, R. H.; Conway, S. J. In vitro photo-release of a TRPV1 agonist. Bioorg. Med. Chem. Lett. 2006, 16, 208-212.
(7) Van Ryssen, M. P.; Avlonitis, N.; Giniatullin, R.; McDougall, C.; Carr, J. L.; Stanton-Humphreys, M. N.; Borgstroem, E. L. A.; Brown, C. T. A.; Fayuk, D.; Surin, A.; Niittykoski, M.; Khiroug, L.; Conway, S. J. Synthesis, photolysis studies and in vitro photorelease of caged TRPV1 agonists and antagonists. Org. Biomol. Chem. 2009, 7, 4695-4707.
(8) Zhao, J.; Gover, T. D.; Muralidharan, S.; Auston, D. A.; Weinreich, D.; Kao, J. P. Y. Caged Vanilloid Ligands for Activation of TRPV1 Receptors by 1- and 2-Photon Excitation. Biochemistry 2006, 45, 4915-4926.
(9) Gilbert, D.; Funk, K.; Dekowski, B.; Lechler, R.; Keller, S.; Moehrlen, F.; Frings, S.; Hagen, V. Caged capsaicins: new tools for the examination of TRPV1 channels in somatosensory neurons. ChemBioChem 2007, 8, 89-97.
(10) Katritzky, A. R.; Xu, Y.-J.; Vakulenko, A. V.; Wilcox, A. L.; Bley, K. R. Model Compounds of Caged Capsaicin: Design, Synthesis, and Photoreactivity. J. Org. Chem. 2003, 68, 9100-9104.
(11) Dore, T. M. Multiphoton Phototriggers for Exploring Cell Physiology. In Dynamic Studies in Biology: Phototriggers, Photoswitches, and Caged Biomolecules; Goeldner, M., Givens, R. S., Eds.; Wiley-VCH: Weinheim, Germany, 2005, p 435-459.
(12) Dore, T. M.; Wilson, H. C. Chromophores for the Delivery of Bioactive Molecules with Two-Photon Excitation. In Photosensitive Molecules for Controlling Biological Function; Chambers, J. J., Kramer, R. H., Eds.; Humana Press: New York, 2011, p 57-92.

### Example 3

Dopamine is a small organic molecule in the catecholamine family of compounds. It is the primary agonist of the dopamine receptor, of which five subtypes exist: D₁-D₅ Dopaminergic signaling has been heavily implicated in reward driven learning,¹ the etiology of a number of neurodegenerative diseases,² and functions as the primary oppositional neurotransmitter to serotonin.³ The complexity of dopaminergic signaling arises from the five different subtypes, each of which exhibits a different expression pattern, and while the D₁ like family (D₁ and D₅) activate adenylate cyclase, the D₂ like family (D₂, D₃, and D₄) inhibit adenylate cyclase.⁴ Because of this complexity, a photochemically activatable dopamine would enable a method for the precise spatial and temporal dissection of the roles dopamine plays in neural signaling. A caged dopamine with sensitivity toward 2PE would provide an even greater level of spatial control over ligand release and receptor activation.^{5,6}

### Discussion:

We disclose here the design and synthesis of a photoactivatable dopamine (Scheme 3.1). Studies on the photophysical and photochemical properties are forthcoming. The design of BHQ-Dopamine stems from the fact that dopamine has a catechol functional group that is important for biological activity, and blocking it with a photoremovable protecting group should render dopamine inactive.⁷ We know from our previous work (Zhu & Dore, unpublished) that BHQ can protect phenol and mediate its photochemical release by 1 PE and 2PE processes (Scheme 3.2). BHQ-OPh is synthesized in two steps from MOM-BHQ-OMs⁸⁻¹⁰ a known compound. BHQ-OPh absorbs light in the UV A region of the spectrum (λₘₐₓ=369, ε=3200 M⁻¹ cm⁻¹) and in neutral buffered aqueous solutions (pH 7.2 KMOPS) undergoes I-photon photolysis at 365 nm with a quantum efficiency Qᵤ=0.19 and 2-photon photolysis uncaging action cross section δᵤ=0.56 GM at 740 nm. BHQ-OPh is stable in the dark under simulated physiological conditions: time constant for hydrolysis in the dark τ_{dark}=95 h. We expect that BHQ-Dopamine will have properties similar to BHQ-OPh, BHQ-O-5HT, and BHQ-caged capsaicinoids.⁸⁻¹⁰

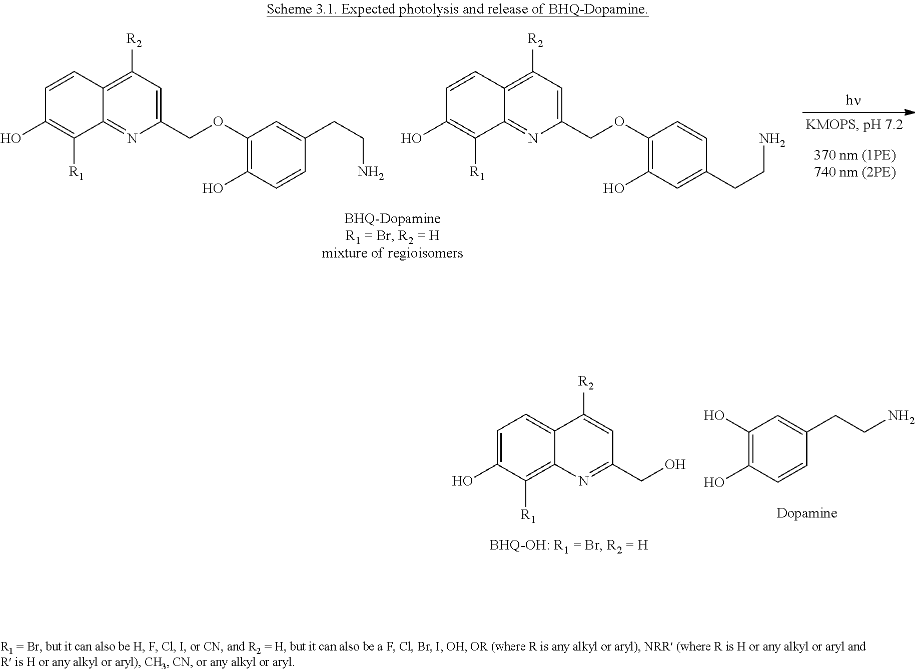

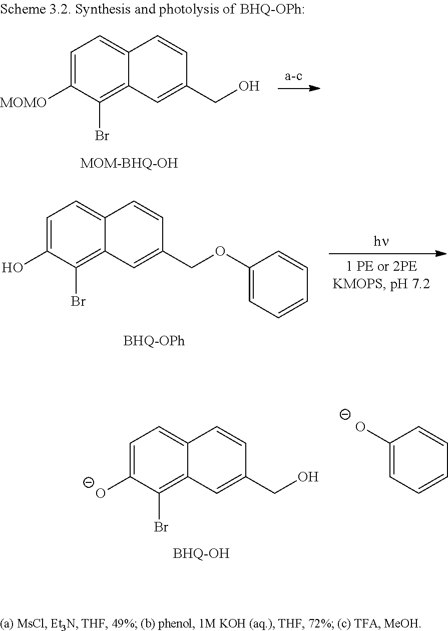

### Preparation of BHQ-Dopamine:

BHQ-Dopamine was prepared as a mixture of regioisomers as shown in Scheme 3.3. Starting from the known compound MOM-BHQ-OMs,⁸⁻¹⁰ the mesylate was displaced by Boc-protected dopamine, which was synthesized according to literature procedure.¹¹ Global deprotection with TMSC1 in MeOH revealed BHQ-Dopamine. Alternative strategies involve using different protecting groups. The MOM group can also be β-methoxyethoxymethyl ether (MEM), methyl (Me), methyl thiomethyl (MTM), benzyloxymethyl (BOM), tetrahydropyranyl (THP), ethoxyethyl (EE), trityl (Tr), methoxytrityl, benzene sulfonyl (Bs), toluenesulfonyl (Ts), or any silicon-based protecting group (e.g., TBS, TBDPS, TIPS). The mesylate (OMs) can also be I, Br, or Cl.

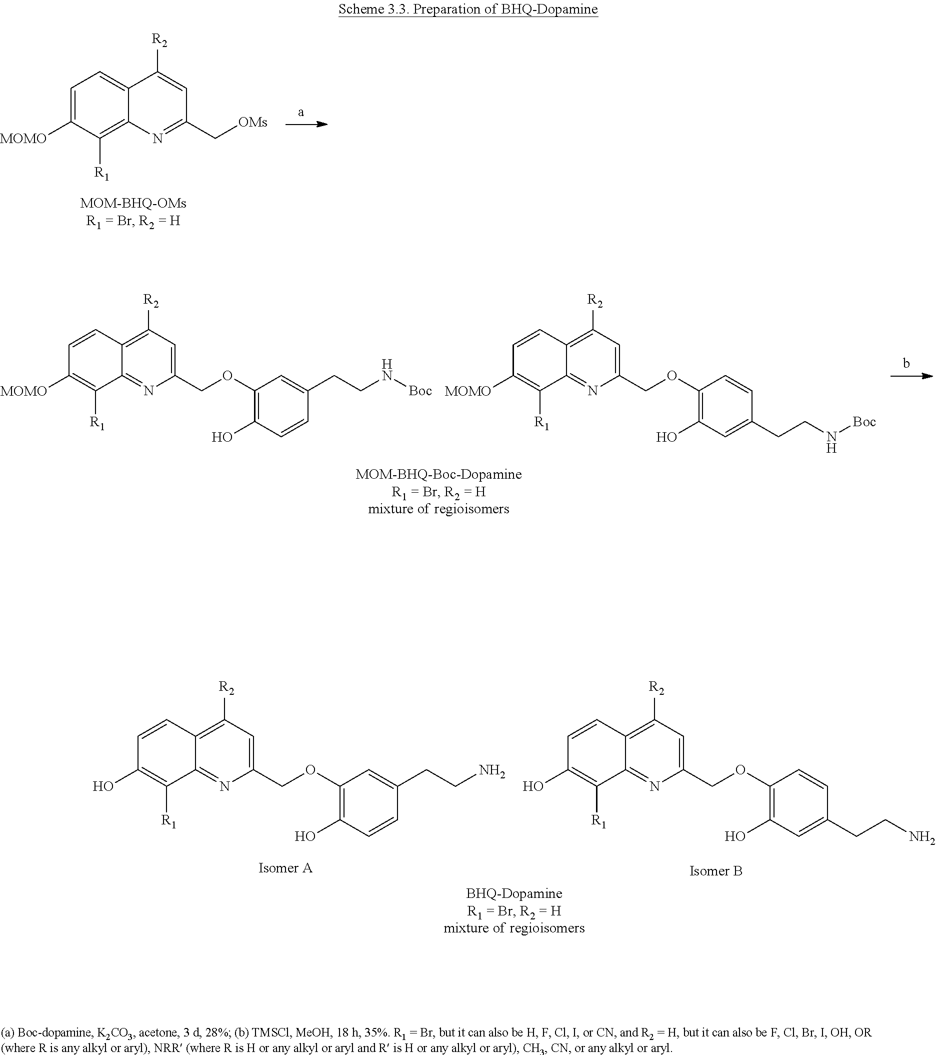

Procedure for preparing BHQ-Dopamine:

MOM-BHQ-Boc-Dopamine:

MOM-BHQ-OMs (0.396 g, 1.06 mmol) was dissolved in acetone. Boc-Dopamine (0.269 g, 1.06 mmol) and potassium carbonate (0.293 g, 2.13 mmol) were added and the reaction stirred at rt for 3 d. The reaction was concentrated in vacuo and the residue purified over silica gel eluting with EtOAc/hexanes to yield the product as a yellow oil (0.176 g, 0.331 mmol): ¹H NMR (400 MHz, CD₃OD) δ 8.39 (d, J=8.3 Hz, 1H), 7.95 (d, J=9.0 Hz, 1H), 7.53 (dd, J=12.3, 8.8 Hz, 2H), 6.76-6.57 (m, 3H), 5.54 (s, 2H), 5.43 (s, 2H), 3.52 (s, 3H), 3.13 (t, J=6.9 Hz, 2H), 2.57 (t, J=7.5 Hz, 2H), 1.48 (s, 9H); MS-ESI (m/z) calcd for [M+H]⁺533.1, 535.1. found 533.0, 535.0.

BHQ-Dopamine:

MOM-BHQ-Boc-Dopamine (0.176 g, 0.33 mmol) was dissolved in methanol and TMSC1 (0.13 mL, 0.99 mmol) was added and the reaction stirred at rt for 18 h. The reaction was concentrated in vacuo to provide a mixture of BHQ-dopamine regioisomers (0.048 g, 0.116 mmol, 35%), which could be separated by HPLC (25:75 CH₃CN/H₂O containing 0.1% TFA) with elution times of 9.1 and 12.2 min. Isomers were distinguished through their respective ROESY spectra. Isomer A: ¹H NMR (400 MHz, CD₃OD) δ 8.20 (d, 1H), 7.64 (d, 1H), 7.41 (d, 1H), 7.16 (d, 1H), 6.87 (s, 1H), 6.80 (d, 2H), 6.71 (d, 2H), 3.06 (q, 2H), 2.73 (t, 2H); ¹³C NMR (101 MHz, CD₃OD) δ 159.1, 156.2, 147.3, 145.8, 137.3, 131.2, 127.9, 123.3, 121.9, 119.5, 118.4, 117.1, 115.9, 114.7, 105.9, 72.1, 40.7, 32.7; HRMS-ESI (m/z) calcd for [M+H]⁺389.0496, 391.0475 found; 389.0502, 391.0482. Isomer B: ¹H NMR (400 MHz, CD₃OD) δ 8.16 (d, 1H), 7.64 (d, 1H), 7.37 (d, 1H), 7.16 (d, 1H), 6.92 (d, 1H), 6.80 (s, 1H), 6.65 (d, 1H), 5.27 (s, 2H) 3.09 (q, 2H), 2.78 (t, 2H); ¹³C NMR (101 MHz, CD₃OD) δ 159.1, 156.2, 147.3, 145.8, 134.3, 131.2, 125.9, 123.3, 121.9, 119.5, 118.4, 117.1, 115.9, 113.6, 102.9, 73.5, 40.7, 32.7.

### Photochemical Properties of BHQ-Dopamine:

We expect that BHQ-dopamine and its derivatives will have similar properties to other BHQ-phenols, such as BHQ-OPh, BHQ-O-5HT, BHQ-capsaicin, BHQ-VNA, and BHQ-VAA. These compounds all have absorbance maxima at 370 nm with large extinction coefficients, robust stability in the dark, large quantum efficiencies of photolysis, and high 2-photon uncaging action cross-sections.

### Procedures for Measuring the Photochemical Properties of BHQ-Dopamine:

Determination of the Molar Extinction Coefficient (ε).

A weighed portion of BHQ-Dopamine was dissolved in water. A measured aliquot of this solution was withdrawn and placed in KMOPS buffer (3.0 mL) and mixed thoroughly to generate a 100-μM solution of BHQ-Dopamine. The absorbance A of this solution at λₘₐₓ=372 nm was measured. This method was repeated twice with different masses of BHQ-Dopamine. The two absorbencies were averaged and the molar extinction coefficient at λₘₐₓ=372 nm was calculated to be 2702 M⁻¹ cm⁻¹ using the equation A=ε1c, where A is the absorbance, λ is the path length of the cuvette, and c is the concentration of the solution.

Determination of the Fluorescence Emission Maxima)λₑₘ).

Stock solutions of BHQ-Dopamine isomer A and isomer B (10 mM in H₂O) were diluted to 100 μM with KMOPS buffer and mixed thoroughly. The excitation wavelength for each isomer was set to λₑₓ=360 nm. The emission maxima was found to be λₑₘ=500 nm for Isomer A and λₑₘ=498 nm for isomer B.

### REFERENCES, EACH OF WHICH IS INCORPORATED HEREIN BY REFERENCE

(1) Bromberg-Martin, E. S.; Matsumoto, M.; Hikosaka, O. Dopamine in Motivational Control: Rewarding, Aversive, and Alerting. Neuron 2010, 68, 815-834.
(2) Kostrzewa, R. M.; Kostrzewa, J. P.; Brown, R. W.; Nowak, P.; Brus, R. Dopamine receptor supersensitivity: development, mechanisms, presentation, and clinical applicability. Neurotoxic. Res. 2008, 14, 121-128.
(3) Wood, M. D.; Wren, P. B. Serotonin-dopamine interactions: Implications for the design of novel therapeutic agents for psychiatric disorders. In Serotonin-Dopamine Interaction: Experimental Evidence and Therapeutic Relevance; Di Giovanni, G., Di Matteo, V., Esposito, E., Eds.; Elsevier: Amsterdam, 2008; Vol. 172, p 213-230.
(4) Neves, S. R.; Ram, P. T.; Iyengar, R. G protein pathways. Science 2002, 296, 1636-1639.
(5) Dore, T. M. Multiphoton Phototriggers for Exploring Cell Physiology. In Dynamic Studies in Biology: Phototriggers, Photoswitches, and Caged Biomolecules; Goeldner, M., Givens, R. S., Eds.; Wiley-VCH: Weinheim, Germany, 2005, p 435-459.
(6) Dore, T. M.; Wilson, H. C. Chromophores for the Delivery of Bioactive Molecules with Two-Photon Excitation. In Photosensitive Molecules for Controlling Biological Function; Chambers, J. J., Kramer, R. H., Eds.; Humana Press: New York, 2011, p 57-92.
(7) Lee, T. H.; Gee, K. R.; Ellinwood, E. H.; Seidler, F. J. Combining “caged-dopamine” photolysis with fast-scan cyclic voltammetry to assess dopamine clearance and release autoinhibition in vitro. J. Neurosci. Meth. 1996, 67, 221-231.
(8) Dore, T. M.; Lauderdale, J. D.; Rea, A. C. BHQ-O-5HT, BHQ-N-5HT, and Related Compounds, Methods of Making the Same, and Methods of Use Thereof. U.S. Patent Application 61501967, Jun. 28, 2011.
(9) Dore, T. M.; Lauderdale, J. D.; Rea, A. C. BHQ-Capsaicin, BHQ-VNA, or BHQ-VAA, and Related Compounds, Methods of Making the Same, and Methods of Use Thereof. U.S. Patent Application 61511586, Jul. 26, 2011.
(10) Rea, A. C. The Synthesis and Characterization of a Series of Caged Neurotransmitters with Two-Photon Sensitivity for Use In Vivo, Masters Thesis, University of Georgia, 2011.
(11) Dalpiaz, A.; Cacciari, B.; Mezzena, M.; Strada, M.; Scalia, S. Solid lipid microparticles for the stability enhancement of a dopamine prodrug. J. Pharm. Sci. 2010, 99, 4730-4737.

### Example 4

### Examples of 4-Substituted Quinoline Derivatives

4-Chloro and 4-diethylamino quinoline derivatives have been prepared from 4-chloro-7-methoxy-2-methylquinoline, a compound known in the literature,¹ as shown in Scheme 4.1. Alternative strategies involve using different protecting groups. The methoxy group can also be hydroxy, methoxymethyl (MOM), β-methoxyethoxymethyl ether (MEM), methyl thiomethyl (MTM), benzyloxymethyl (BOM), tetrahydropyranyl (THP), ethoxyethyl (EE), trityl (Tr), methoxytrityl, benzene sulfonyl (Bs), toluenesulfonyl (Ts), or any silicon-based protecting group (e.g., TBS, TBDPS, TIPS).

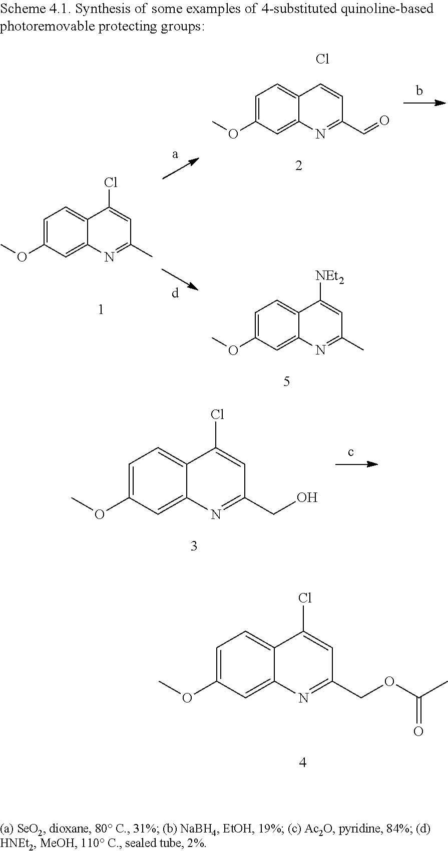

### 4-Chloro-7-methoxyquinoline-2-carbaldehyde (2)

4-Chloro-7-methoxy-2-methylquinoline (1, 128 mg, 0.616 mmol) was added to a flask containing SeO₂ (109 mg, 0.982 mmol) and dioxane (5 mL), and stirred at 80° C. for 5 hours. The mixture was gravity filtered, concentrated, and purified over silica (EtOAc/hexanes) to yield 4-chloro-7-methoxyquinoline-2-carbaldehyde (2) (67.8 mg, 0.306 mmol, 31%) as a white powder: ¹H NMR (400 MHz, CDCl₃) d 10.15 (d, J=1.1 Hz, 1H), 8.20 (d, J=8.9 Hz, 1H), 7.97 (d, J=1.1 Hz, 1H), 7.55 (d, J=2.4 Hz, 1H), 7.43 (dd, J=9.2, 2.5 Hz, 1H), 4.02 (d, J=1.1 Hz, 3H); HRMS-ESI (m/z) calcd for [M+H]⁺222.0316 found; 222.0319.

### (4-Chloro-7-methoxyquinolin-2-yl)methanol (3)

4-Chloro-7-methoxyquinoline-2-carbaldehyde (2, 67.8 mg, 0.306 mmol) was added to a flask containing sodium borohydride (17.4 mg, 0.459 mmol) and ethanol (3 mL), and stirred for 1 hour. The mixture was gravity filtered, concentrated, and purified over silica (EtOAc/hexanes) to yield (4-chloro-7-methoxyquinolin-2-yl)methanol (3, 12.6 mg, 0.057 mmol, 19%) as a white powder: ¹H NMR (400 MHz, CDCl₃) d 8.09 (d, J=9.0 Hz, 1H), 7.39 (d, 1H), 7.26 (m, 2H), 4.86 (s, 2H), 4.97 (s, 4H).

### (4-Chloro-7-methoxyquinolin-2-yl)methyl acetate (4)

(4-Chloro-7-methoxyquinolin-2-yl)methanol (3, 12.6 mg, 0.057 mmol) was added to a flask containing acetic anhydride (0.1 mL, 0.907 mmol) and pyridine (3 mL), and stirred for 1 hour. The mixture was concentrated and purified over silica (EtOAc/hexanes) to yield (4-chloro-7-methoxyquinolin-2-yl)methyl acetate (4, 12.7 mg, 0.0478 mmol) as a white powder: ¹H NMR (400 MHz, CDCl₃) d 8.10 (d, J=9.1 Hz, 1H), 7.43 (s, 1H), 7.41 (d, J=2.5 Hz, 1H), 7.29 (dd, 1H), 5.32 (s, 2H), 3.95 (s, 3H); HRMS-ESI (m/z) calcd for [M+H]⁺266.0578 found; 266.0579.

### N,N-Diethyl-7-methoxy-2-methylquinolin-4-amine (5)

4-Chloro-7-methoxy-2-methylquinoline (1, 83 mg, 0.401 mmol) was added to a bomb reactor containing diethylamine (0.1 mL, 1.93 mmol), and methanol (3 mL). The reactor was heated to 110° C. for 2 hours. The resulting reaction mixture was concentrated in vacuo and purified over silica (EtOAc/hexanes) to yield N,N-diethyl-7-methoxy-2-methylquinolin-4-amine (5) as a white solid (2 mg, 0.0077 mmol, 2%): ¹H NMR (400 MHz, CDCl₃) d 7.88 (d, J=8.9 Hz, 1H), 7.31 (d, J=2.5 Hz, 1H), 7.03 (dd, J=9.2, 2.6 Hz, 1H), 6.63 (s, 1H), 3.93 (m, 3H), 3.33 (q, J=7.1 Hz, 4H), 2.64 (s, 3H), 1.15 (t, J=7.0 Hz, 6H); HRMS-ESI (m/z) calcd for [M+H]⁺245.1648 found; 245.1649.

### Prophetic Compounds for Use with BHQ Phototriggers

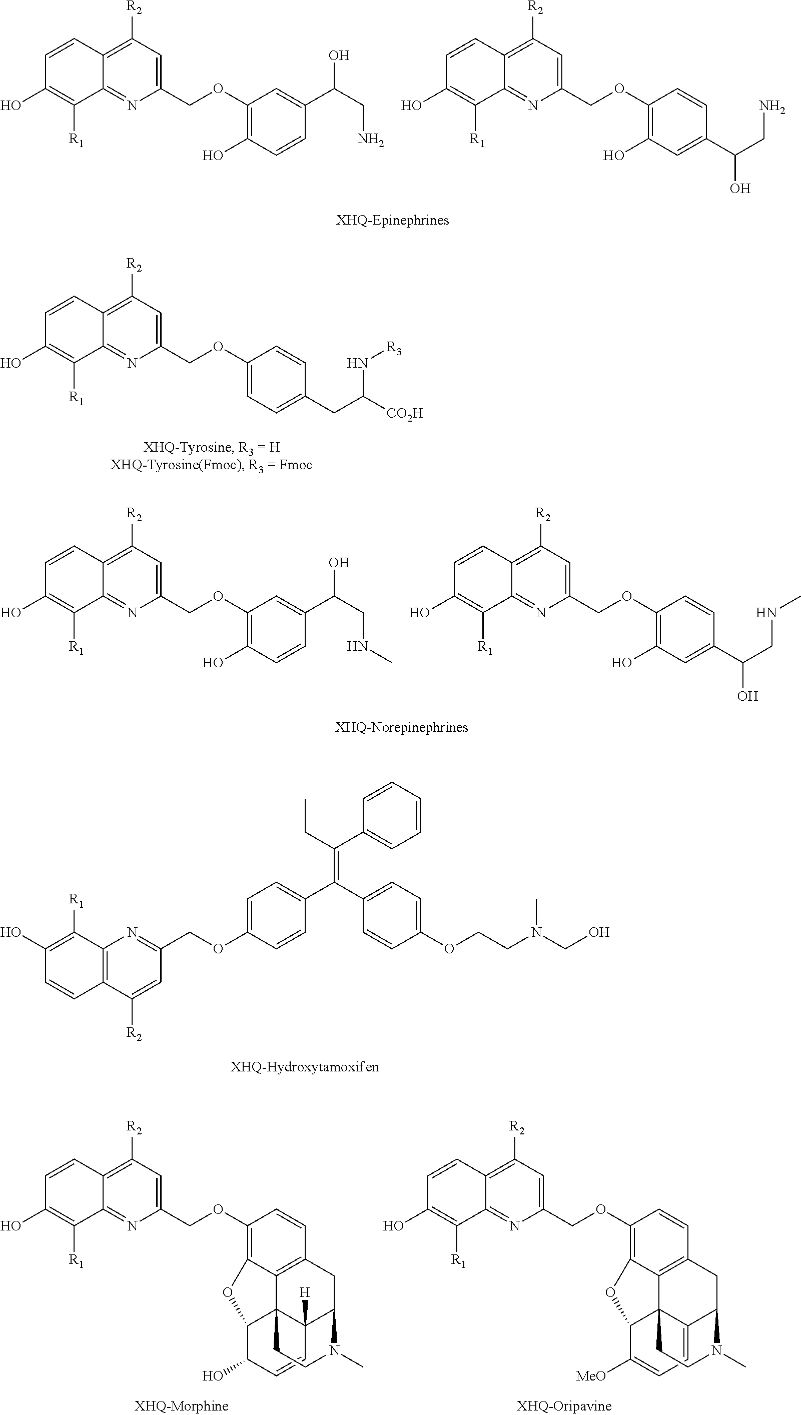

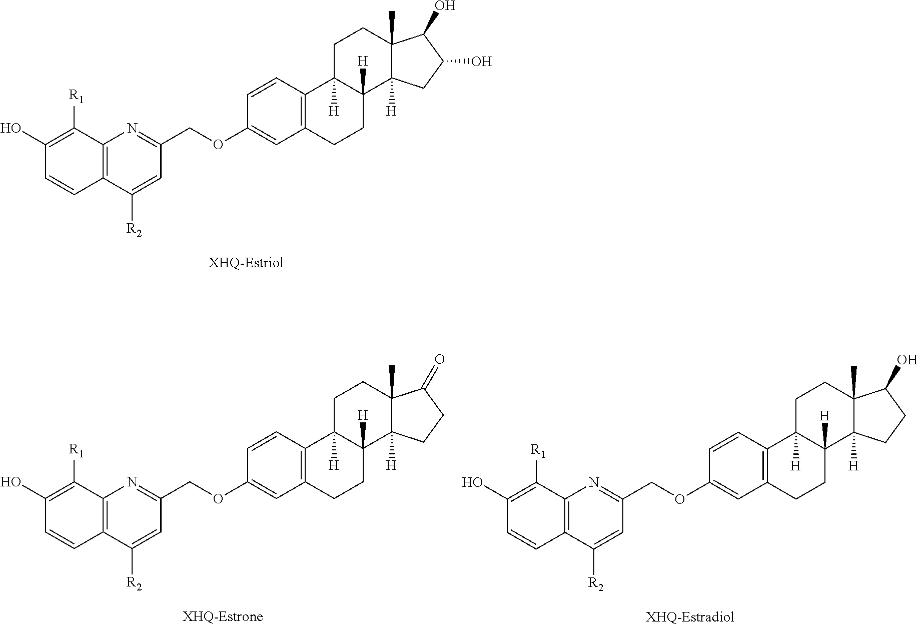

### REFERENCE, WHICH IS INCORPORATED HEREIN BY REFERENCE

(1) Abe, Y.; Kayakiri, H.; Satoh, S.; Inoue, T.; Sawada, Y.; Inamura, N.; Asano, M.; Aramori, I.; Hatori, C.; Sawai, H.; Oku, T.; Tanaka, H. A Novel Class of Orally Active Non-Peptide Bradykinin B2 Receptor Antagonists. 3. Discovering Bioisosteres of the Imidazo[1,2-a]pyridine Moiety. J. Med. Chem. 1998, 41, 4062-4079.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to the values and/or measuring techniques. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

At least the following is claimed:

1. A composition, comprising:
a BHQ-conjugate or a protected BHQ-conjugate precursor compound.

2. The composition of claim 1, wherein the conjugate is selection from the group consisting of: a serotonin (5HT), a capsaicinoid, and a catechol.

3. The composition of claim 1, wherein the BHQ-conjugate is selected from the group consisting of: BHQ-O-5HT, BHQ-N-5HT, BHQ-capsaicin, BHQ-VNA (vanillyamide of n-nonanoic acid), BHQ-VAA, BHQ-dopamine, BHQ-epinephrine, BHQ-noreepinephrine, BHQ-tyrosine, BHQ-tyrosine(Fmoc), BHQ-hydroxytamoxifen, BHQ-morphine, BHQ-oripavine, BHQ-estriol, BHQ-estrone, and BHQ-estradiol.

4. The composition of claim 1, wherein the BHQ-conjugate has the following structure: 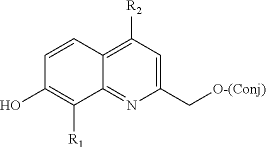
wherein R₁ is selected from the group consisting of: H, Br, F, Cl, I, and CN; and wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl.

5. The composition of claim 1, wherein the protected BHQ-conjugate precursor compound has the following structure: 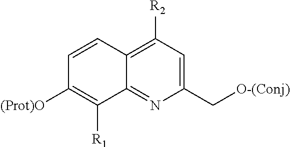
wherein R₁ is selected from the group consisting of: H, Br, F, Cl, I, and CN; wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl; wherein the Prot group is selected from the group consisting of: a methoxymethyl ether (MOM) group, a β-methoxyethoxymethyl ether (MEM) group, a methyl group (Me), a methyl thiomethyl (MTM) group, a benzyloxymethyl (BOM) group, a tetrahydropyranyl (THP) group, an ethoxyethyl (EE) group, a trityl (Tr) group, a methoxytrityl group, a benzene sulfonyl (Bs) group, a toluenesulfonyl (Ts) group, and a silicon-based protecting group.

6. A method of treating a condition, comprising administering a pharmaceutically effective amount of BHQ-conjugate to a subject in need of treatment.

7. The method of claim 6, wherein the conjugate is selection from the group consisting of: a serotonin (5HT), a capsaicinoid, and a catechol.

8. The method of claim 6, wherein the BHQ-conjugate has the following structure: 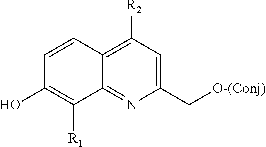
wherein R₁ is selected from the group consisting of: H, Br, F, Cl, I, and CN; and wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl.

9. A method of releasing a conjugate, comprising:
exposing a BHQ-conjugate to a light energy, wherein the light energy interacts with the BHQ-conjugate and causes the conjugate to be released from the BHQ-conjugate.

10. The method of claim 9, wherein the light energy is about 300 to 425 nm or 690-850 nm.

11. The method of claim 9, wherein the BHQ-conjugate has the following structure: 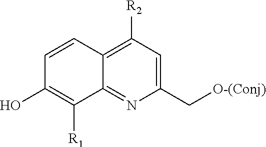
wherein R₁ is selected from the group consisting of: H, Br, F, Cl, I, and CN; and wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl.

12. A pharmaceutical composition, comprising:
a pharmaceutically effective amount of a BHQ-conjugate.

13. The pharmaceutical composition of claim 12, wherein the conjugate is selection from the group consisting of: a serotonin (5HT), a capsaicinoid, and a catechol.

14. The pharmaceutical composition of claim 12, wherein the BHQ-conjugate is selected from the group consisting of: BHQ-O-5HT, BHQ-N-5HT, BHQ-capsaicin, BHQ-VNA (vanillyamide of n-nonanoic acid), BHQ-VAA, BHQ-dopamine, BHQ-epinephrine, BHQ-noreepinephrine, BHQ-tyrosine, BHQ-tyrosine(Fmoc), BHQ-hydroxytamoxifen, BHQ-morphine, BHQ-oripavine, BHQ-estriol, BHQ-estrone, and BHQ-estradiol.

15. The pharmaceutical composition of claim 12, wherein the BHQ-conjugate has the following structure: 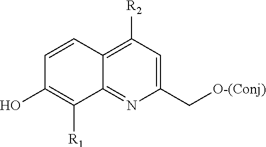
wherein R₁ is selected from the group consisting of: H, Br, F, Cl, I, and CN; and wherein R₂ is selected from the group consisting of: H, F, Cl, Br, I, OH, OR, NRR′, CH₃, CN, an unsubstituted or substituted alkyl, and unsubstituted or substituted aryl, wherein R and R′ are each independently selected from the group consisting of: H, an unsubstituted or substituted alkyl, and unsubstituted or substituted aryl.
